(19) United States
(12) Patent Application Publication
(10) Pub. No.: US 2022/0333178 A1
(43) Pub. Date: Oct. 20, 2022

(54) METHODS FOR IMPROVING NUCLEIC ACID CLUSTER CLONALITY

(71) Applicants: Illumina Cambridge Limited, Cambridge (GB); Illumina, Inc., San Diego, CA (US)

(72) Inventors: Gary Mark Skinner, Kedington (US); Geraint Evans, Cambridge (GB); Niall Gormley, Cambridge (GB); Jonathan Boutell, Bishops Stortford (GB); Matthew W. Kellinger, San Diego, CA (US); Michael Previte, San Diego, CA (US); Molly He, San Diego, CA (US)

(21) Appl. No.: 17/699,573

(22) Filed: Mar. 21, 2022

Related U.S. Application Data
(60) Provisional application No. 63/164,163, filed on Mar. 22, 2021.

(51) Int. Cl. C12Q 1/6848; C12Q 1/70

(52) U.S. Cl. CPC C12Q 1/6848; C12Q 1/70; C12Q 1/6869

(57) ABSTRACT

A method for seeding and amplifying target nucleic acids derived from a sample in a cluster at a site on a surface of a substrate includes retaining at least a portion of the target nucleic acids in an inactive form that cannot seed to provide a relatively low concentration of active form target nucleic acids available for seeding. As the active form target nucleic acids seed on the surface of the substrate, they may be amplified. Because the concentration of active form target nucleic acids is low, the likelihood is low that a second active form target nucleic acid will seed at the same site within the same cluster before the first active form target nucleic acid is sufficiently amplified to dominate. Accordingly, the likelihood that the cluster will pass filters is increased relative to traditional seeding and amplification methods employing a higher concentration of active form target nucleic acids.

### RELATED APPLICATIONS

This application claims priority to Provisional Application No. 63/164,163 filed Mar. 22, 2021 entitled METHOD FOR IMPROVING NUCLEIC ACID CLUSTER CLONALITY the contents of which is incorporated herein by reference in its entirety.

### FIELD

The present disclosure relates to, among other things, amplifying target nucleic acids to generate clusters; and more particularly to increasing the number of clusters that are monoclonal or have high percent dominance.

### BACKGROUND

Sequencing of a target nucleic acid strand may occur through multiple cycles of reactions by which one detectable nucleotide per cycle is incorporated into a copy strand. The detectable nucleotides are typically blocked to prevent incorporation of more than one detectable nucleotide per cycle. After an incubation time, a wash step is typically performed to remove any unincorporated detectable nucleotide. A detection step, in which the identity of the detectable nucleotide incorporated into the copy strand is determined, may then be performed. Next, an unblocking step and cleavage or masking step is performed in which the blocking agent is removed from the last incorporated nucleotide in the copy strand and the detectable moiety is cleaved from or masked on the last nucleotide incorporated into the copy strand. In some instances, the detectable moiety serves as the blocking agent, and removal of the detectable moiety may remove the blocking agent. The cycle is then repeated by introducing detectable nucleotides in a subsequent incorporation step.

In many cases, clusters of target nucleic acid strands having the same sequence are simultaneously sequenced. The clusters serve to amplify the signal produced by detectable nucleotides incorporated into the copy strands. Because the clusters contain multiple template strands of the same sequence, the nucleotide incorporated into the corresponding copy strands at each round of nucleotide addition should be the same, and the signal from the detectable nucleotide should be enhanced proportional to the number of copies of the template strand in the cluster.

Clusters of target nucleic acid strands may be formed on a substrate such as a solid surface by, for example, contacting a sample including a plurality of target nucleic acids under conditions sufficient for a target nucleic acid to hybridize with a capture primer on a surface of the substrate in a step referred to as seeding. The seeded target nucleic acid may be amplified to produce the cluster. The capture primer may be one of a pair of primers bound to the surface of the substrate to allow for bridge amplification. The capture primers may be limited to particular locations of the substrate, such as wells, on a patterned flow cell, or the like, to isolate amplified colonies from one another.

In some cases, the clusters are polyclonal rather than monoclonal. Polyclonal clusters may result from amplification of more than one target nucleic acid in a cluster. If polyclonal clusters have a single target species that is present at a concentration sufficiently higher than other target species such that the signal from the dominant species can be resolved from the noise of the non-dominant species in a cluster, the dominant target species may be sequenced. A target species may become dominant because it seeds and amplifies prior to seeding and amplification of one or more subsequent non-dominant species.

A polyclonal cluster that contains a dominate target species that produces a signal sufficiently above the background of other species and thus may be sequenced is said to be “passing filters” or PF. Currently employed technology and processes for high throughput sequencing provide for a PF in range of 60% to 80%, which means that 20% to 40% of the clusters are not sequenceable. To increase throughput and increase sequencing efficiency, it is desirable to increase the percentage of PF clusters.

One way to increase the percentages of clusters that PF is to reduce the concentration of the target nucleic acids in the sample contacted with the substrate to generate the clusters. By reducing the concentration of the target nucleic acids, chances are reduced that more than one nucleic acid will attach to the primer on the surface and will be amplified. However, reducing the concentration of the target nucleic acids in the sample also increases the likelihood that some cluster sites will not be seeded and will not contain an amplified nucleic acid for sequencing.

Instead of a single seed and amplification step with a sample having low concentration of target nucleic acids, separate seed and amplification steps may be performed to achieve higher dominancy and PF of clusters with more cluster locations being occupied. In each step, a much lower DNA template concentration may be used so that only a fraction of the locations become seeded, but each of these amplifies to be much less polyclonal, with higher dominancy, and is therefore much more likely to PF. Through multiple rounds of seeding and amplification more locations of the substrate may be occupied by clusters of target nucleic acid.

However, a process that include multiple seed/amplification steps has several drawbacks. For example, the process is more complicated than a process that includes only a single seed/amplification step, takes more time than a process that includes only a single seed/amplification step, and may use more amplification reagents than a process that includes only a single seed/amplification step.

### SUMMARY

The present application describes, among other things, a method for seeding and amplifying target nucleic acids derived from a sample to form a cluster at a site on a surface of a substrate. At least a portion of the target nucleic acids are initially in an inactive form that cannot seed, leaving a relatively low concentration of active form target nucleic acids available for seeding. As the active form target nucleic acids seed on the surface of the substrate, they may be amplified. Because the concentration of active form nucleic acids is low, the likelihood is low that a second active form target nucleic acid will seed at the same site and within the same cluster before the first active form target nucleic acid has sufficiently amplified to dominate. Accordingly, the likelihood that the cluster will PF is increased relative to traditional seeding and amplification methods employing a higher concentration of nucleic acids.

While it is possible to perform more than one seeding and amplification step using the initially inactivate form nucleic acids as described herein, a single seeding and amplification step may provide some of the benefits of multi-step low concentration seeding and amplification without the drawbacks of multiple steps. A composition comprising the target nucleic acids may be contacted with the substrate for a sufficient time to allow most or all of the sites to be seeded and amplified to form clusters that are monoclonal or have a high percent dominance.

In various aspects, the present disclosure describes a method comprising providing a substrate having a surface to which a capture agent is bound and providing a composition comprising (i) a plurality of different target nucleic acids, each comprising a universal sequence, and (ii) an inhibiting agent that inhibits binding of at least a portion of the universal sequence to the capture agent. The method further includes contacting the surface of the substrate with the composition to bind one of the target nucleic acids to the capture agent. The method may also include amplifying the target nucleic acid bound to the capture agent while the composition is in contact with the surface of the substrate

The capture agent bound to the surface of the substrate may comprise a nucleic acid having a nucleotide sequence. In some embodiments, the universal sequence comprises a nucleotide sequence complementary to at least a portion of the nucleotide sequence of the nucleic acid of the capture agent.

The inhibiting agent may comprise a nucleic acid having a nucleotide sequence that is the same as at least the portion of the nucleotide sequence of the nucleic acid of the capture agent. The composition further comprises an unblocking agent that comprises a nucleic acid having a nucleotide sequence complementary to at least a portion of the nucleic acid of the inhibiting agent. The concentration of the unblocking agent in the composition is lower than the concentration of the inhibiting agent.

In some embodiments, the inhibiting agent encapsulates the plurality of different target nucleic acids. For example, the inhibiting agent may comprise a liposome or a phage that encapsulates the target nucleic acids. The composition may further comprise an unblocking agent configured to release the plurality of different target nucleic acids encapsulated in the inhibiting agent. For example, when the inhibiting agent comprises a liposome, the unblocking agent may comprise a molecule configured to disrupt a membrane of the liposome to release the target nucleic acids from the liposome. For example, the unblocking agent may comprise a porin, talin, or a cytoskeletal submembranous protein. By way of another example, when the inhibiting agent comprises a bacteriophage λ, the unblocking agent may comprise lamB.

In various aspects, the present disclosure describes a method comprising providing a substrate having a surface to which a capture agent is bound and providing a composition comprising a plurality of different target nucleic acids, each comprising a universal sequence configured to bind the capture agent. The universal sequence of at least some of the plurality of different nucleic acids are blocked from binding to the capture agent. At least some of the nucleic acids are blocked from binding to the binding sites of the surface of the substrate. The method further comprises contacting the surface of the substrate with the composition and unblocking the universal sequence of at least some of the plurality of different target nucleic acids to allow the unblocked nucleic acids to bind to the capture agent. The method may further include amplifying the nucleic acids that are bound to the capture agent while the composition is in contact with the surface of the substrate.

In some embodiments, the composition in the methods described herein is configured to provide a concentration of target nucleic acids available to bind to the capture agents in a range from about 5 picomolar (pM) to about 50 pM during a time in which the composition is contacted with the surface of the substrate. The total concentration of nucleic acids in the composition, including those that are available to bind the binding sites and those that are not available to bind the binding sites may, in some embodiments, be in a range from about 50 pM to about 1 nM.

The plurality of different target nucleic acids may comprise DNA, such a library DNA.

The capture agent may be a part of an array of capture agents.

The substrate may be part of a sequencing flow cell.

### Definitions

Terms used herein will be understood to take on their ordinary meaning in the relevant art unless specified otherwise. Several terms used herein, and their meanings are set forth below.

As used herein, the term “array” refers to a population of sites that may be differentiated from each other according to relative location. Different molecules that are at different sites of an array may be differentiated from each other according to the locations of the sites in the array. An individual site of an array may include one or more molecules of a particular type. For example, a site may include a single target nucleic acid molecule having a particular sequence or a site may include several nucleic acid molecules having the same sequence (and/or complementary sequence, thereof). The sites of an array may be defined by features on a substrate or an apparatus. Illustrative features include without limitation, wells, beads (or other particles), projections from a surface, ridges on a surface, a patterned coating on a surface, or channels in a surface. For example, each site of an array may be defined by a well.

As used herein, the term “amplicon,” when used in reference to a nucleic acid, means the product of copying the nucleic acid, wherein the product has a nucleotide sequence that is the same as or complementary to at least a copied portion of the nucleotide sequence of the nucleic acid. An amplicon may be produced by any of a variety of amplification methods that use the nucleic acid, e.g., a target nucleic acid or an amplicon thereof, as a template including, for example, polymerase extension, polymerase chain reaction (PCR), rolling circle amplification (RCA), ligation extension, or ligation chain reaction. An amplicon may be a nucleic acid molecule having a single copy of a particular nucleotide sequence (e.g. a polymerase extension product) or multiple copies of the nucleotide sequence (e.g. a concatemeric product of RCA). A first amplicon of a target nucleic acid is typically a complementary copy. Subsequent amplicons are copies that are created, after generation of the first amplicon, from the target nucleic acid or from the first amplicon. A subsequent amplicon may have a sequence that is substantially complementary to the target nucleic acid or substantially identical to the target nucleic acid.

As used herein, the term “capacity,” when used in reference to a site and a nucleic acid, means the maximum amount of nucleic acid, e.g., amplicons derived from a target nucleic acid, that may occupy the site. For example, the term may refer to the total number of nucleic acids that may occupy the site in a particular condition. Other measures may be used as well including, for example, the total mass of nucleic acid or the total number of copies of a particular nucleotide sequence that may occupy the site in a particular condition. Typically, the capacity of a site for a target nucleic acid will be substantially equivalent to the capacity of the site for amplicons of the target nucleic acid.

As used herein, the term “amplification site” refers to a site of an array where one or more amplicons may be generated. An amplification site may be further configured to contain, hold or attach at least one amplicon that is generated at the site. An amplification site may comprise a capture agent.

As used herein, the term “capture agent” refers to a material, chemical, molecule, or moiety thereof that is capable of attaching or retaining to a target molecule (e.g. a target nucleic acid). Illustrative capture agents include, without limitation, a capture nucleic acid that is complementary to at least a portion of a target nucleic acid (e.g., modified to include a universal capture binding sequence), a member of a receptor-ligand binding pair (e.g. avidin, streptavidin, biotin, lectin, carbohydrate, nucleic acid binding protein, epitope, antibody, etc.) capable of binding to a modified target nucleic acid (or linking moiety attached thereto), or a chemical reagent capable of forming a covalent bond with a modified target nucleic acid (or linking moiety attached thereto). In some embodiments, a capture agent comprises a nucleic acid. In some embodiments, a capture agent comprising a nucleic acid may be used as an amplification primer.

For purposes of the present specification, “binding” of a target nucleic acid to a capture agent means that the target nucleic acid is attached or retained relative to the capture agent in a manner suitable to carry out an operation on the bound target nucleic acid. For example, the bound target nucleic acid may be sufficiently attached or retained relative to the capture agent to carry out amplification of the target nucleic acid while the target nucleic acid remains attached or retained to the capture agent under the amplification conditions. Binding may include covalent binding, hybridization, and the like. Binding has a similar meaning regarding target nucleic acids and inhibiting agents, and the like. For purposes of the present specification, a nucleic acid that is “seeded” on a substrate is bound to the substrate; e.g. via a capture agent.

The terms “P5” and “P7” may be used when referring to a nucleic acid capture agent. The terms “P5′” (P5 prime) and “P7” (P7 prime) refer to the complements of P5 and P7, respectively. It will be understood that any suitable nucleic acid capture agent may be used in the methods presented herein, and that the use of P5 and P7 are illustrative embodiments only. Uses of nucleic acid capture agents such as P5 and P7 on flow cells is known in the art, as exemplified by the disclosures of WO 2007/010251, WO 2006/064199, WO 2005/065814, WO 2015/106941, WO 1998/044151, and WO 2000/018957. One of skill in the art will recognize that a nucleic acid capture agent may also function as an amplification primer. For example, any suitable nucleic acid capture agent may act as a forward amplification primer, whether immobilized or in solution, and may be useful in the methods presented herein for hybridization to a sequence (e.g., a universal capture binding sequence) and amplification of a sequence. Similarly, any suitable nucleic acid capture agent may act as a reverse amplification primer, whether immobilized or in solution, and may be useful in the methods presented herein for hybridization to a sequence (e.g., a universal capture binding sequence) and amplification of a sequence. In view of the general knowledge available and the teachings of the present disclosure, one of skill in the art will understand how to design and use sequences that are suitable for capture and amplification of target nucleic acids as presented herein.

As used herein, the term “universal sequence” refers to a region of sequence that is common to two or more target nucleic acids, where the molecules also have regions of sequence that differ from each other. A universal sequence that is present in different members of a collection of molecules may allow capture of multiple different nucleic acids using a population of capture nucleic acids that are complementary to a portion of the universal sequence, e.g., a universal capture binding sequence. Non-limiting examples of universal capture binding sequences include sequences that are identical to or complementary to P5 and P7 primers. P5 has the following nucleotide sequence: AAT GAT ACG GCG ACC ACC GA (SEQ ID NO:1). P7 has the following nucleic acid sequence: CAA GCA GAA GAC GGC ATA CGA GAT (SEQ ID NO:2). A universal sequence present in different members of a collection of molecules may allow the replication or amplification of multiple different nucleic acids using a population of universal primers that are complementary to a portion of the universal sequence, e.g., a universal primer binding site. Target nucleic acids may be modified to attach universal adapters (also referred to herein as adapters), for example, at one or both ends of the different target sequences, as described herein.

As used herein, the term “adapter” and its derivatives, e.g., universal adapter, refers generally to any linear oligonucleotide which may be ligated to a target nucleic acid. In some embodiments, the adapter is substantially non-complementary to the 3′ end or the 5′ end of any target sequence present in a sample. In some embodiments, suitable adapter lengths are in the range of about 10-100 nucleotides, about 12-60 nucleotides and about 15-50 nucleotides in length. Generally, the adapter may include any combination of nucleotides and/or nucleic acids. The adapter may include one or more cleavable groups at one or more locations. The adapter may include a sequence that is substantially identical, or substantially complementary, to at least a portion of a primer, for example a capture agent comprising a nucleic acid. The adapter may include a barcode, also referred to as an index or tag, to assist with downstream error correction, identification, or sequencing.

As defined herein, “sample” and its derivatives are used in its broadest sense and includes any specimen, culture or the like that is suspected of including a target nucleic acid. In some embodiments, the sample comprises one or more of DNA, RNA, PNA, LNA, chimeric or hybrid forms of nucleic acids. The sample may include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample may be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material may include nucleic acids obtained from a newborn, for example as typically used for newborn screening.

As used herein, the term “double stranded,” when used in reference to a nucleic acid molecule, means that nucleotides in the nucleic acid molecule are hydrogen bonded to a complementary nucleotide. A partially double stranded nucleic acid may have at least 10%, at least 25%, at least 50%, at least 60%, at least 70%, at least 80%, at least 90% or at least 95% of its nucleotides hydrogen bonded to a complementary nucleotide.

As used herein, the term “each,” when used in reference to a collection of items, is intended to identify an individual item in the collection but does not necessarily refer to every item in the collection unless the context clearly dictates other.

As used herein, the term “excluded volume” refers to the volume of space occupied by a particular molecule to the exclusion of other such molecules.

As used herein, the term “interstitial region” refers to an area in a substrate or on a surface that separates other areas of the substrate or surface. For example, an interstitial region may separate one site of an array from another site of the array. The two sites that are separated from each other may be discrete, lacking contact with each other. In another example, an interstitial region may separate a first portion of a site from a second portion of a site. The separation provided by an interstitial region may be partial or full separation. Interstitial regions will typically have a surface material that differs from the surface material of the sites on the surface. For example, sites of an array may have an amount or concentration of capture agents that exceeds the amount or concentration present at the interstitial regions. In some embodiments the interstitial regions may be free of capture agents.

As used herein, the term “polymerase” is intended to be consistent with its use in the art and includes, for example, an enzyme that produces a complementary replicate of a nucleic acid molecule using the nucleic acid as a template strand. Typically, DNA polymerases bind to the template strand and then move down the template strand sequentially adding nucleotides to the free hydroxyl group at the 3′ end of a growing strand of nucleic acid. DNA polymerases typically synthesize complementary DNA molecules from DNA templates and RNA polymerases typically synthesize RNA molecules from DNA templates (transcription). Polymerases may use a short RNA or DNA strand, called a primer, to begin strand growth. Some polymerases may displace the strand upstream of the site where they are adding bases to a chain. Such polymerases are said to be strand displacing, meaning they have an activity that removes a complementary strand from a template strand being read by the polymerase. Illustrative polymerases having strand displacing activity include, without limitation, the large fragment of Bsu (Bacillus subtilis), Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases are modified, either by mutation or otherwise, to reduce or eliminate 3′ and/or 5′ exonuclease activity.

As used herein, the term “nucleic acid” is intended to be consistent with its use in the art and includes naturally occurring nucleic acids and functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure may have an alternate backbone linkage including any of a variety of those known in the art. Naturally occurring nucleic acids generally have a deoxyribose sugar (e.g. found in deoxyribonucleic acid (DNA)) or a ribose sugar (e.g. found in ribonucleic acid (RNA)). A nucleic acid may contain any of a variety of analogs of these sugar moieties that are known in the art. A nucleic acid may include native or non-native bases. In this regard, a native deoxyribonucleic acid may have one or more bases selected from adenine, thymine, cytosine or guanine and a ribonucleic acid may have one or more bases selected from uracil, adenine, cytosine or guanine. Useful non-native bases that may be included in a nucleic acid are known in the art. Nucleic acids may comprise two or more nucleotides.

The term “target,” when used in reference to a nucleic acid, is intended as a semantic identifier for the nucleic acid in the context of a method or composition set forth herein and does not necessarily limit the structure or function of the nucleic acid beyond what is otherwise explicitly indicated. A target nucleic acid having a universal sequence at each end, for instance a universal adapter at each end, may be referred to as a modified target nucleic acid. For purposes of the present disclosure, “target nucleic acid” and “modified target nucleic acid” are used interchangeably and refer to any nucleic acid having a target nucleotide sequence.

As used herein, the term “transport” refers to movement of a molecule through a fluid. The term may include passive transport such as movement of molecules along their concentration gradient (e.g. passive diffusion). The term may also include active transport whereby molecules may move along their concentration gradient or against their concentration gradient. Thus, transport may include applying energy to move one or more molecule in a desired direction or to a desired location such as an amplification site.

As used herein, the term “rate,” when used in reference to transport, amplification, capture or other chemical processes, is intended to be consistent with its meaning in chemical kinetics and biochemical kinetics. Rates for two processes may be compared with respect to maximum rates (e.g. at saturation), pre-steady state rates (e.g. prior to equilibrium), kinetic rate constants, or other measures known in the art. A rate for a particular process may be determined with respect to the total time for completion of the process. For example, an amplification rate may be determined with respect to the time taken for amplification to be complete. However, a rate for a particular process need not be determined with respect to the total time for completion of the process.

The term “and/or” means one or all of the listed elements or a combination of any two or more of the listed elements. “Or” is used herein to mean “and/or” unless context dictates otherwise. The use of “and/or” in some situations does not mean that the use of “or” in other situations is intended to not mean “and/or.”

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “comprises” and variations thereof do not have a limiting meaning where these terms appear in the description and claims.

It is understood that wherever embodiments are described herein with the language “comprises,” “include,” “includes,” or “including,” and the like, otherwise analogous embodiments described in terms of “consisting of” and/or “consisting essentially of” are also provided. For example, a substrate that comprises a bead may be a substrate that consists of or consists essentially of a bead.

Unless otherwise specified, “a,” “an,” “the,” and “at least one” are used interchangeably and mean one or more than one.

Conditions that are “suitable” for an event to occur, such as hybridization of two nucleic acid sequences, or “suitable conditions” are conditions that do not prevent such events from occurring. Thus, these conditions permit, enhance, facilitate, and/or are conducive to the event.

As used herein, “providing” in the context of a composition, an article, or a nucleic acid means making the composition, article, or nucleic acid, purchasing the composition, article, or nucleic acid, or otherwise obtaining the compound, composition, article, or nucleic acid.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” “some embodiments,” “one aspect,” “an aspect,” “certain aspects,” or “some aspects,” etc., means that a particular feature, configuration, composition, or characteristic described in connection with the embodiment or aspect is included in at least one embodiment or aspect of the disclosure. Thus, the appearances of such phrases in various places throughout this specification are not necessarily referring to the same embodiment of the disclosure. The appearances of such phrases in various places through this specification do not necessarily mean that the separately mentioned aspects or embodiments are not the same or combinable. Particular features, configurations, compositions, or characteristics disclosed herein may be combined in any suitable manner in one or more embodiments or aspects.

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### BRIEF DESCRIPTION OF THE DRAWINGS

The following detailed description of illustrative embodiments of the present disclosure may be best understood when read in conjunction with the following drawings.

FIG. 1 is a schematic top-down plan view of an embodiment of a flow cell comprising an array.

FIG. 2 is a schematic side plan view of an embodiment of a portion of a site of an array, showing capture agents attached to the surface of a substrate.

FIG. 3 is a schematic side plan view of the site of the array of FIG. 2, showing a target nucleic acid bound to a capture agent.

FIG. 4 is a schematic overview of an embodiment of a mechanism for maintaining a low concentration of active form target nucleic acids for seeding and amplifying on a surface of a site of an array. A pool of inactive form target nucleic acids is converted to active form at a rate that maintains a low concentration of active form target nucleotide to increase the likelihood that only a single target nucleic acid species seeds on the surface of the site and to increase the likelihood that a monoclonal cluster is generated by amplification.

FIG. 5 is a schematic overview of an embodiment of a mechanism for maintaining a low concentration of active form target nucleic acids in a seeding composition. A pool of inactive form target nucleic acids is converted to active form at a rate that maintains a low concentration of active form target nucleotide. The rate at which the inactive form is converted to an active form target nucleic acid is largely determined by the rate of interaction of an unblocking agent with a component of the inactive form.

FIG. 6 is a schematic drawing of an embodiment of an example of a mechanism for converting an inactive form target nucleic acid to an active form nucleic acid in a seeding composition at a rate that increases the likelihood that only a single active form target nucleic acid will seed to the surface of a substrate at a site of an array. A portion of the inactive form target nucleic acid is blocked by an inhibitory agent to prevent seeding. Conversion to the active form occurs intrinsically in the seeding composition at a suitable rate to maintain a low concentration of the active form target nucleic acid.

FIG. 7 is a schematic drawing of an embodiment of an example of a mechanism for converting an inactive form target nucleic acid to an active form nucleic acid in a seeding composition. A portion of the inactive form target nucleic acid is blocked by an inhibitory agent to prevent seeding. An unblocking agent interacts with the inhibitory agent to release the active form of the target nucleic acid. The rate of conversion from the inactive form to the active form of the target nucleic acid is largely controlled by the rate of the interaction of the unblocking agent with the inhibitory agent.

FIG. 8 is a schematic drawing of an embodiment of an example of a mechanism for converting an inactive form target nucleic acid to an active form nucleic acid in a seeding composition. The inactive form target nucleic acids are encapsulated in a vehicle such as a liposome. An unblocking agent interacts with the vehicle to cause the release of the active form target nucleic acid. The rate of conversion from the inactive form to the active form of the target nucleic acid is largely controlled by the rate of the interaction of the unblocking agent with the vehicle.

FIG. 9 is a schematic drawing of an embodiment of an example of a mechanism for converting an inactive form target nucleic acid to an active form nucleic acid in a seeding composition. The inactive form target nucleic acids are encapsulated in a phage. An unblocking agent, such as LamB, interacts with the phage to cause the release of the active form target nucleic acid. The rate of conversion from the inactive form to the active form of the target nucleic acid is largely controlled by the rate of the interaction of the unblocking agent with the phage.

FIG. 10 is a schematic drawing of an embodiment of an example of a mechanism for converting an inactive form target nucleic acid to an active form nucleic acid in a seeding composition at a rate that increases the likelihood that only a single active form target nucleic acid will seed to the surface of a substrate at a site of an array. The target nucleic acid has an extension that forms a hairpin loop and blocks the target nucleic acid from binding to a capture agent on a substrate at a site of an array. Conversion to the active form occurs intrinsically in the seeding composition at a suitable rate to maintain a low concentration of the active form target nucleic acid.

FIG. 11 is a schematic drawing of an embodiment of an example of a mechanism for converting an inactive form target nucleic acid to an active form nucleic acid in a seeding composition at a rate that increases the likelihood that only a single active form target nucleic acid will seed to the surface of a substrate at a site of an array. The target nucleic acid has an extension that forms a hairpin loop and blocks the target nucleic acid from binding to a capture agent on a substrate at a site of an array. A restriction enzyme may cleave the extension to facilitate conversion to the active form at an appropriate rate.

FIG. 12 is a stochastic optical reconstruction microscopy (STORM) image of 550 nm pitch, 360 nm diameter nanowell clusters. Different colors emanated from many of the clusters, indicating that the clusters were polyclonal.

FIG. 13 is a graph of the relationship of % PF and % Dominancy. The graph shows that as clusters increase in % Dominancy, they also increase in % PF. Each data point represents the mean % Dominancy determined by STORM imaging and mean % PF, determined by sequencing, of a population of several 10s to 100s of nanowells.

FIG. 14A is a series of STORM images from a repeat seed/amp experiment of nanowells. In this experiment, there were 5 sequential steps performed from left to right, in the first 4 steps (Red, Green, White, Magenta), 20 pM DNA was incubated with amplification mix for 15 minutes, then flushed with buffer. In the final step, 200 pM DNA was seeded and amped to fill in the final wells (Yellow).

FIG. 14B is a graph showing results obtained from the images depicted in FIG. 14A. The repeat seed/amp clusters were analyzed by STORM imaging to determine their dominancy, showing that this strategy may shift the dominancy distribution to higher % Dominancy. A higher % PF was confirmed by sequencing.

FIGS. 15A and 15B are images illustrating phage mediated controlled release of DNA library molecules into solution, ready for seeding. DNA is held securely within the phage capsid (15A) until the addition of the LamB trigger protein caused phage capsid to release the DNA into solution (15B). FIG. 15A shows some DNA held on the surface within the phage, and FIG. 15B shows the same area some time later after LamB caused DNA molecules to be released from the phage.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION OF ILLUSTRATIVE EMBODIMENTS

The present application relates to, among other things, methods for seeding or seeding and amplifying target nucleic acids derived from a sample in a cluster at a site on a surface of a substrate. At least a portion of the target nucleic acids are initially in an inactive form that cannot seed, leaving a relatively low concentration of active form target nucleic acids available for seeding. The active form target nucleic acids may be amplified as they seed on the surface of the substrate. Because the concentration of active form target nucleic acids is low, the likelihood is low that a second active form target nucleic acid will seed at the same site before the first active form target nucleic acid is sufficiently amplified to dominate. By keeping the concentration of active form target nucleic acids low, the rate of seeding of a nucleic acid on a surface of the substrate may be kept low relative to the rate of amplification of the seeded nucleic acid, which may allow the first seeded nucleic acid to be amplified to an extent sufficient to dominate prior to a second nucleic acid seeding in the same site. Accordingly, the likelihood that the cluster at a site will PF is increased relative to traditional seeding and amplification methods employing higher concentrations of active nucleic acids.

Arrays

The methods described herein may be employed to seed target nucleic acids on any suitable substrate. Preferably, the substrate, or an apparatus comprising one or more substrates, has an array of capture agents. A capture agent of the array preferably serves as at least a portion of an amplification site.

The substrate may comprise any suitable material that may include, or may be modified to include, a capture agent. Examples of suitable material include glass, modified glass, functionalized glass, inorganic glasses, microspheres (e.g. inert and/or magnetic particles), plastics, polysaccharides, nylon, nitrocellulose, ceramics, resins, silica, silica-based materials, carbon, metals, an optical fiber or optical fiber bundles, polymers and multiwell (e.g. microtiter) plates. Illustrative plastics include acrylics, polystyrene, copolymers of styrene and other materials, polypropylene, polyethylene, polybutylene, polyurethanes and Teflon. Illustrative silica-based materials include silicon and various forms of modified silicon.

In some embodiments, the substrate is within or part of a larger apparatus, such as a well, tube, channel, cuvette, Petri plate, bottle or the like. Preferably, a flow cell comprises the substrate. Examples of suitable flow cells include an eight-channel flow cell used in the cBot sequencing workstation (Illumina, Inc., San Diego, Calif.) and those described in, for example, U.S. Pat. No. 8,241,573, WO 2007/123744, or Bentley et al., Nature 456:53-59 (2008). Illustrative flow cells include those commercially available from Illumina, Inc. (San Diego, Calif.). Optionally, the flow cell is a patterned flow cell. Suitable patterned flow cells include, but are not limited to, flow cells described in WO 2008/157640. In some embodiments, a microwell plate or a microtiter plate comprises the substrate.

The capture agents may be located at any suitable site of an array on the substrate or apparatus comprising one or more substrates.

In some embodiments, the sites of an array may be configured as features on a surface of the substrate. The features may be present in any of a variety of desired formats. For example, the sites may comprise wells, pits, channels, ridges, raised regions, pegs, posts or the like. The sites may comprise beads or particles. In some embodiments, the sites do not comprise beads or particles. Illustrative sites include wells that are present in apparatuses comprising substrates used for commercial sequencing platforms sold by 454 LifeSciences (a subsidiary of Roche, Basel Switzerland) or Ion Torrent (a subsidiary of Life Technologies, Carlsbad Calif.). Other apparatus comprising wells include, for example, etched fiber optics and other substrates described in U.S. Pat. Nos. 6,266,459; 6,355,431; 6,770,441; 6,859,570; 6,210,891; 6,258,568; 6,274,320; 8,262,900; 7,948,015; U.S. Pat. Pub. No. 2010/0137143; U.S. Pat. No. 8,349,167, or PCT Publication No. WO 00/63437. In several cases the substrates are exemplified in these references for applications that use beads as the substrate in a well. The well-containing substrates may be used with or without beads in the methods or compositions of the present disclosure. In some embodiments, wells of a substrate may include gel material (with or without beads) as set forth in U.S. Pat. No. 9,512,422.

The sites of an array may comprise metal features on a non-metallic surface such as glass, plastic or other materials described above. A metal layer may be deposited on a surface using methods known in the art such as wet plasma etching, dry plasma etching, atomic layer deposition, ion beam etching, chemical vapor deposition, vacuum sputtering, or the like. Any of a variety of commercial instruments may be used as appropriate including, for example, the FlexAL®, OpAL®, Ionfab 300Plus®, or Optofab 3000® systems (Oxford Instruments, UK). A metal layer may also be deposited by e-beam evaporation or sputtering as set forth in Thornton, Ann. Rev. Mater. Sci. 7:239-60 (1977). Metal layer deposition techniques, such as those exemplified above, may be combined with photolithography techniques to create metal regions or patches on a surface. Illustrative methods for combining metal layer deposition techniques and photolithography techniques are provided in U.S. Pat. Nos. 8,778,848 and 8,895,249.

An array of features, which may define sites of the array, may appear as a grid of spots or patches. The features may be located in a repeating pattern or in an irregular non-repeating pattern. Particularly useful patterns are hexagonal patterns, rectilinear patterns, grid patterns, patterns having reflective symmetry, patterns having rotational symmetry, or the like. Asymmetric patterns may also be useful. The pitch may be the same between different pairs of nearest neighbor features or the pitch may vary between different pairs of nearest neighbor features. In particular embodiments, features of an array may each have an area that is larger than about 100 nm², 250 nm², 500 nm², 1 μm², 2.5 μm², 5 μm², 10 μm², 100 μm², or 500 μm². Alternatively, or additionally, features of an array may each have an area that is smaller than about 1 mm², 500 μm², 100 μm², 25 μm², 10 μm², 5 μm², 1 μm², 500 nm², or 100 nm². Indeed, a region may have a size that is in a range between an upper and lower limit selected from those exemplified above.

For embodiments that include an array of features defining sites on a surface, the features may be discrete, being separated by interstitial regions. The size of the features and/or spacing between the sites may vary such that arrays may be high density, medium density or low density. High density arrays may have regions separated by less than about 15 μm. Medium density arrays have regions separated by about 15 to 30 μm, while low density arrays have regions separated by greater than 30 μm. An array useful for methods and apparatuses described herein may have low-density arrays, medium-density arrays, or high-density arrays. In some embodiments, the apparatuses having arrays as described herein have regions that are separated by less than 100 μm, 50 μm, 10 μm, 5 μm, 1 μm or 0.5 μm.

In some embodiments, an array may include a collection of beads or other particles. The particles may be suspended in a solution or they may be located on the surface of a substrate. Examples of bead arrays in solution are those commercialized by Luminex (Austin, Tex.). Examples of arrays having beads located on a surface include those wherein beads are located in wells such as a BeadChip array (Illumina Inc., San Diego Calif.) or substrates used in sequencing platforms from 454 LifeSciences (a subsidiary of Roche, Basel Switzerland) or Ion Torrent (a subsidiary of Life Technologies, Carlsbad Calif.). Other arrays having beads located on a surface are described in U.S. Pat. Nos. 6,266,459; 6,355,431; 6,770,441; 6,859,570; 6,210,891; 6,258,568; 6,274,320; US 2009/0026082 A1; US 2009/0127589 A1; US 2010/0137143 A1; US 2010/0282617 A1 or PCT Publication No. WO 00/63437. Several of the above references describe methods for attaching target nucleic acids to beads prior to loading the beads in or on an array substrate. It will be understood that the beads may be made to include capture agents and the beads may then be used to load an array, thereby forming amplification sites for use in a method set forth herein. As set forth previously herein, the substrates may be used without beads. For example, capture agents may be attached directly to wells or to gel material in wells. Thus, the references are illustrative of materials, compositions or apparatus that may be modified for use in the methods and compositions set forth herein.

Amplification sites of an array may include a plurality of capture agents capable of binding to target nucleic acids. In some embodiments, a capture agent includes a capture nucleic acid. The nucleotide sequence of the capture nucleic acid may be complementary to a sequence of one or more target nucleic acids, such as a sequence of a region of a universal adapter. In some embodiments, the capture nucleic acid may function as a primer for amplification of the target nucleic acid. In some embodiments, one population of capture nucleic acid includes a P5 primer or the complement thereof, and the second population of capture nucleic acid includes a P7 primer or the complement thereof.

A capture agent, such as a capture nucleic acid, may be attached to the amplification site. For example, the capture agent may be attached to the surface of a feature defining a site of an array. The attachment may be via an intermediate structure such as a bead, particle or gel. An example of attachment of capture nucleic acids to an array via a gel is described in U.S. Pat. No. 8,895,249 and further exemplified by flow cells available commercially from Illumina Inc. (San Diego, Calif.) or described in WO 2008/093098. Illustrative gels that may be used in the methods and apparatus set forth herein include, but are not limited to, those having a colloidal structure, such as agarose; polymer mesh structure, such as gelatin; or cross-linked polymer structure, such as polyacrylamide, SFA (see, for example, US Pat. App. Pub. No. 2011/0059865 A1) or PAZAM (see, for example, U.S. Prov. Pat. App. Ser. No. 61/753,833 and U.S. Pat. No. 9,012,022). Attachment via a bead may be achieved as exemplified in the description and cited references set forth previously herein.

In some embodiments, the features defining sites on the surface of an array are non-contiguous, being separated by interstitial regions of the surface. Interstitial regions that have a substantially lower quantity or concentration of capture agents, compared to the features of the array, are advantageous. Interstitial regions that lack capture agents are particularly advantageous. For example, a relatively small amount or absence of capture moieties at the interstitial regions favors localization of target nucleic acids, and subsequently generated clusters, to desired features. In some embodiments, the features may be concave features in a surface (e.g. wells) and the features may contain a gel material. The gel-containing features may be separated from each other by interstitial regions on the surface where the gel is substantially absent or, if present the gel is substantially incapable of supporting localization of nucleic acids. Methods and compositions for making and using substrates having gel containing features, such as wells, are set forth in U.S. Pat. No. 9,512,422.

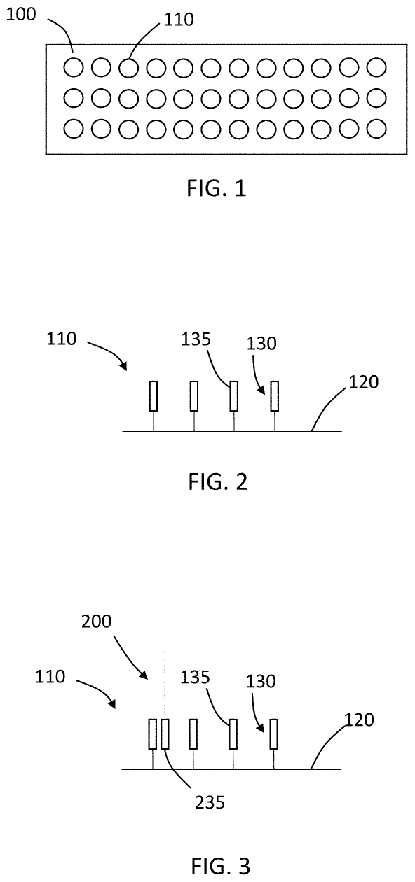

An example of a flow cell 100 having an array comprising a plurality of sites 110 is shown schematically in FIG. 1. In the depicted embodiment, the sites 110 comprise wells that may contain capture agent. For example, FIGS. 2 and 3 illustrate a portion of a site 110 of an array, which may be a well as depicted in FIG. 1. FIG. 2 shows the site 110 with no hybridized target nucleic acid, and FIG. 3 shows the site 110 with a hybridized target nucleic acid 200. The site 110 includes a substrate surface 120 and a plurality of capture agents 130 on the surface 120. The capture agents 130 comprise a sequence of nucleotides 135 complementary to a portion 235 of a target nucleic acid 200. For example, capture agent 130 may comprise a P5 or a P7 primer sequence and the target nucleic acid 200 may comprise a universal adapter sequence that comprises at its 3′ end a nucleotide sequence complementary to at least a portion of the P5 or P7 primer sequence (e.g., sequence of nucleotides 135). When the flow cell 100 is contacted with a composition comprising a library of target nucleic acids at the site 110, the portion 235 of the target nucleic acid 200 may hybridize with the complementary sequence of nucleotides 135 of the capture agent 130. The capture agent 130 may serve as an amplification primer to copy the target nucleic acid 200. For example, the capture agent 130 may comprise a free 3′ end from which extension may occur using the target nucleic acid 200 as a template. Accordingly, the site 110 may be an amplification site on which a cluster may be developed.

While not shown, it will be understood that that a second capture agent may be on the surface of the site to allow for bridge amplification. In such instances, the 5′ end of the target nucleic acid may include a universal adapter having a sequence that is the same as a sequence of nucleotides of the second capture agent. Accordingly, when the target nucleic acid is copied using the first capture agent 130 as a primer, the resulting copied nucleic acid will have a complementary sequence that may hybridize to the second capture agent during bridge amplification.

Seeding Composition

Seeding may be accomplished by contacting the sites of the array with a composition comprising target nucleic acids. The composition is preferably a liquid composition at temperatures employed for seeding. The composition may be a solution, suspension, dispersion, or the like. Preferably, the nucleic acids are fully dissolved in the composition.

The seeding composition may include one or more components for amplifying a seeded target nucleic acid. For example, appropriate nucleotides, buffers, enzymes, cofactors, primers, or the like may be included in the seeding compositions.

The seeding composition may comprise any suitable concentration of total target nucleic acid. For example, the seeding composition may comprise from about 50 pM to about 1 nM total target nucleic acid, such as from about 100 pM to about 700 pM, from about 200 pM to about 500 pM, or about 300 pM total target nucleic acid. Preferably, the seeding composition is formed from a target nucleic acid library comprising numerous different target nucleic acids.

The seeding composition may have any suitable concentration of target nucleic acid in an active form capable of binding a capture agent at a site of an array. For example, the concentration of active form target nucleic acid may be in a range from about 5 pM to about 50 pM, such as from about 10 pM to about 40 pM, from about 15 pM to about 30 pM, or about 20 pM.

As indicated above, keeping the concentration of active form target nucleic acid low during seeding reduces the likelihood that more than one target nucleic acid will seed at a particular site, and thus increases the likelihood that a cluster resulting from amplification at the site will be monoclonal or will have a higher percent dominance than with traditional seeding and amplification employing higher concentrations of active form target nucleic acids. With traditional seeding and amplification, all, or substantially all, the target nucleic acids in a seeding composition are active form target nucleic acids.

Any suitable mechanism for providing a low concentration of active form target nucleic acids from a pool of higher concentration total target nucleic acids may be employed. For example, an agent may inhibit binding of at least a portion of a target nucleic acid with a capture agent and seeding on a surface of a site of an array for cluster formation. The inhibiting agent may encapsulate one or more target nucleic acids or may block a portion of a target nucleic acid to prevent seeding on the surface. When the inhibiting agent inhibits binding of the target nucleic acid to the capture agent, the target nucleic acid is an inactive form.

The inhibiting agent preferably selectively interacts with the target nucleic acid, such as a universal sequence of a target nucleic acid rather than inhibiting binding through non-selective mechanisms such as molecular crowding or the like.

An equilibrium may exist between blocking of the target nucleic acid by the inhibiting agent (inactive form) and release or unblocking of the target nucleic acid from the inhibiting agent (active form). Preferably the equilibrium favors a lower concentration of active form target nucleic acid than of inactive form nucleic acid. The released or unblocked active form target nucleic acid may bind the capture agent at a site of an array (i.e., seed on the surface) and be amplified to form a cluster. Equilibrium between inactive form and active form target nucleic acids may be intrinsic within a seeding composition.

In some embodiments, such equilibrium may be depicted as indicated below in Formula I:

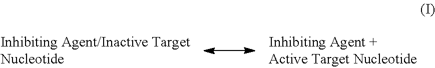

In some embodiments, the seeding composition comprises an unblocking agent that interacts with the inhibiting agent to release or unblock the target nucleic acid and convert the target nucleic acid from inactive form to active form. The unblocking agent may be incorporated in a seeding composition just prior to contacting the seeding composition is contacted with the array or while the seeding composition is in contact with the array.

An equilibrium may exist between interaction of the inhibiting agent and the unblocked agent, which may drive the concentration of active form target nucleic acid in a composition. For example, if the target nucleic acid is in inactive form when it is associated with the inhibiting agent but is in active form when the inhibiting agent is associated with the unblocking agent, equilibrium between free unblocking agent and unblocking agent associated with inhibiting agent is preferably shifted towards free unblocking agent.

In some embodiments, such equilibrium may be depicted as indicated below in Formula II:

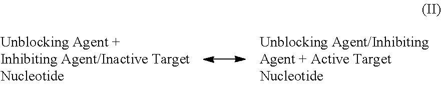

In some embodiments, the conversion of target nucleic acids from inactive form to active form is effectively irreversible or substantially irreversible in a seeding composition. In such embodiments, the rate of conversion from inactive form to active form is preferably slower than the rate amplification. The rate of conversion of an inactive form target nucleic acid to an active form target nucleic acid may largely control the rate of seeding. Conversion from inactive form to active form may be intrinsic (e.g., similar to Formula I, but in one direction) or may be driven by an unblocking agent (e.g., similar to Formula II, but in one direction).

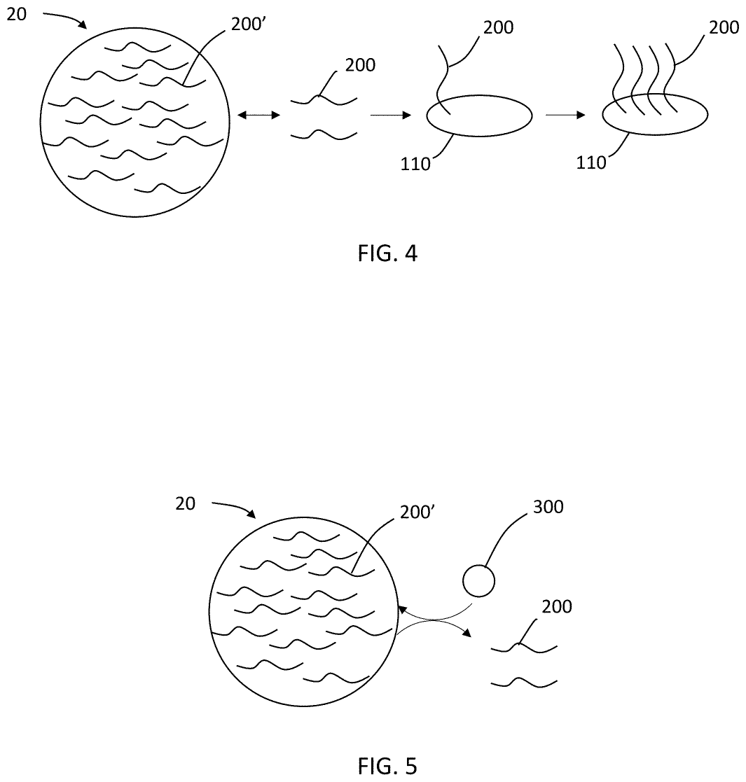

FIGS. 4 and 5 schematically show mechanisms for providing a pool 20 of nucleic acid having a relatively high concentration of inactive form target nucleic acids 200′ to achieve a relatively low concentration of active form target nucleic acids 200. The inactive form target nucleic acids 200′ do not seed on a surface of a site of an array, while the active form target nucleic acids 200 may seed (e.g., via interaction with a capture agent) on a surface of a site 110 of an array. The seeded target nucleic acid 200 may be amplified to form a cluster (e.g., as shown at the far right of FIG. 4). Conversion from inactive form to active form may be intrinsic (e.g., as shown in FIG. 4) or may be driven by an unblocking agent 300 (e.g., as shown in FIG. 5).

Any suitable inhibiting agent 200 may be used to inhibit binding of at least a portion of a target nucleic acid with the capture agent. The inhibiting agent may be an extension of the target nucleic acid or may be separate from the target nucleotide. The inhibiting agent may encapsulate one or more target nucleic acids. The inhibiting agent may block a portion of the target nucleic acid that binds to a capture agent when unblocked.

Inhibiting Agent Separate from Target Nucleic Acid

A. Blocking

Any suitable inhibiting agent that blocks the target nucleic acid from binding the capture agent at a site of an array may be used. At least a portion of the blocked nucleic acids (inactive form) may be converted to the active form. This may occur intrinsically in the seeding composition or may be driven by an unblocking agent.

The inhibiting agent may comprise a nucleic acid that hybridizes to a portion of the target nucleic acid configured to bind the capture agent and thus inhibit binding of the target nucleotide to the capture agent. The inhibiting agent may be a nucleic acid of any length. For example, the inhibiting agent may have a length of about 10 nucleotides or more, such as 100 nucleotides or more, or 1000 nucleotides or more. The inhibiting agent may be a nucleic acid having a length greater than the target nucleic acid. The inhibiting agent preferably has a length less than the target nucleic acid. For example, the inhibiting agent may have a length of about 10 to about 100 nucleotides, about 12 to about 60 nucleotides, or about 15 to about 50 nucleotides.

At least a portion of the nucleic acid inhibiting agent may hybridize with the portion of the target nucleic acid configured to bind the capture agent. In some embodiments, the inhibiting agent comprises a nucleotide sequence that is fully complementary to the entire portion of the target nucleic acid configured to bind the capture agent. In some embodiments, the inhibiting agent comprises a nucleotide sequence that is not fully complementary to the entire portion of the target nucleic acid configured to bind the capture agent. The inhibiting agent may comprise a nucleotide sequence that is complementary to a nucleotide sequence that is 5′ or 3′ to the portion of the target nucleic acid configured to bind the capture agent.

The percent complementarity, length of complementarity, or region of complementarity of the inhibitory agent to the target nucleic acid may be adjusted to tailor the relative affinity of the capture agent and the inhibitory agent for the target nucleic acid, which may tailor the relative concentration of inactive form target nucleic acids and active form target nucleic acids available for seeding via binding to the capture agent. In addition, the complementary portions may include non-natural nucleotides, such as locked nucleotides, that may enhance or reduce stability of base pair binding between the inhibitory agent and the target nucleic acid to alter the relative affinity of the capture agent and the inhibitory agent for the target nucleic acid.

The concentration of the nucleic acid inhibitory agent in the seeding composition may be adjusted to achieve a desired ratio of inactive target nucleic acid with hybridized inhibitory agent and active form nucleic acid that it not hybridized with the inhibitory agent by shifting equilibrium. The concentration of nucleic acid inhibitory agent to achieve the desired concentration of active form target nucleic acid may depend on the concentration of total target nucleic acid; the percent complementarity, length of complementarity, or region of complementarity of the inhibitory agent to the target nucleic acid; the sequence of the region of complementarity; and whether the inhibitory agent or target nucleic acid includes any non-natural nucleotides that enhance or reduce base pair binding stability relative to the natural nucleotide counterpart.

The concentration of the nucleic acid inhibitory agent may be greater than, equal to, or less than the total concentration of target nucleic acid. Preferably, the concentration of the nucleic acid inhibitory agent is greater than the concentration total target nucleic acid in the seeding composition. For example, the concentration of the nucleic acid inhibitory agent may be about 1.5 times or more greater than the concentration of total target nucleic acid, amount 2 times or more greater than the concentration of total target nucleic acid, or about 2.5 times or more greater than the concentration of total target nucleic acid.

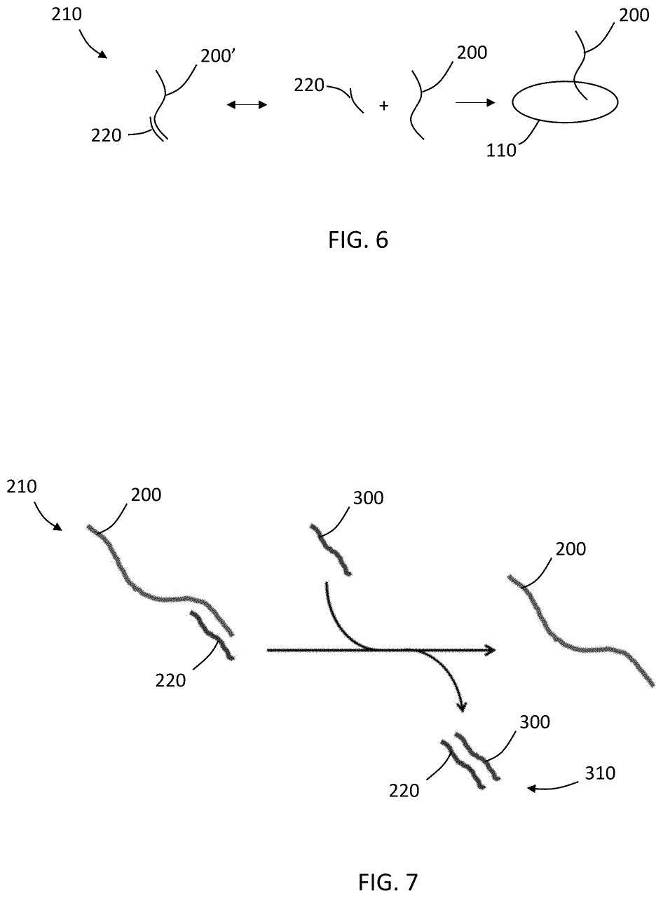

FIG. 6 schematically shows conversion of an inactive form target nucleic acid 200′ that is complexed 210 with an inhibitory nucleic acid agent 220 to an active form target nucleic acid 200 that may seed on a surface of a site 110 of an array (e.g., via hybridizing with a capture agent). The conversion occurs intrinsically in the seeding solution to maintain a low concentration of active form target nucleic acid 200 to reduce likelihood that more than one target nucleic acid 200 seeds on the site 110 of the array such that, when amplified, a resulting cluster is monoclonal or has a high percent dominancy.

In some embodiments, the seeding composition comprises a nucleic acid inhibitory agent and a nucleic acid unblocking agent. The inhibitory nucleic acid agent may be as described above. The nucleotide unblocking agent may have a nucleic acid sequence that competes with the target nucleic acid for binding to the inhibitory agent. When the unblocking agent is hybridized to the inhibitory agent, the target nucleic acid is released and is in active form.

At least a portion of the nucleic acid unblocking agent may hybridize with the nucleic acid inhibitory agent. In some embodiments, the unblocking agent comprises a nucleotide sequence that is fully complementary to the entire inhibitory agent. In some embodiments, the unblocking agent comprises a nucleotide sequence that is not fully complementary to the entire inhibitory agent. In some embodiments, the unblocking agent has the same sequence as the portion of the capture agent to which the target nucleic acid may hybridize.

Preferably, the inhibitory agent is partially complementary to the target nucleic acid and the unblocking agent has a higher degree of complementary to the inhibitory agent than the inhibitory agent has to the target nucleic acid. In some embodiments, the inhibitory agent is partially complementary to the target nucleic acid and the unblocking agent is fully complementary to the inhibitory agent.

The percent complementarity, length of complementarity, or region of complementarity of the inhibitory agent to the target nucleic acid and of the unblocking agent to the inhibitory agent may be adjusted to tailor the relative affinity of the capture agent and the inhibitory agent for the target nucleic acid and the unblocking agent for the inhibitory agent, which may tailor the relative concentration of inactive form target nucleic acids and active form target nucleic acids available for seeding via binding to a capture agent. In addition, the complementary portions may include non-natural nucleotides, such as locked nucleotides, that may enhance or reduce stability of base pair binding between the inhibitory agent and the target nucleic acid and the unblocking agent and the inhibitory agent to alter the relative affinity of the capture agent and the inhibitory agent for the target nucleic acid and the unblocking agent for the inhibitory agent.

The concentration of the nucleic acid inhibitory agent and the nucleic acid unblocking agent in the seeding composition may be adjusted to achieve a desired ratio of inactive target nucleic acid and active form nucleic acid. The concentration of inhibitory agent and the unblocking agent to achieve the desired concentration of active form target nucleic acid may depend on the concentration of total target nucleic acid; the percent complementarity, length of complementarity, or region of complementarity of the inhibitory agent to the target nucleic acid; the percent complementarity, length of complementarity, or region of complementarity of the unblocking agent to the inhibitory agent; the sequence of the region of complementarity between the inhibitory agent and the target nucleic acid and the sequence of the region of complementarity between the unblocking agent and the inhibitory agent; and whether the inhibitory agent, unblocking agent or target nucleic acid includes any non-natural nucleotides that enhance or reduce base pair binding stability relative to the natural nucleotide counterpart.

Preferably, the concentration of the nucleic acid inhibitory agent is greater than the concentration total target nucleic acid in the seeding composition and the concentration of the nucleic acid unblocking agent is less than the concentration than the total target nucleic acid in the seeding composition. In some embodiments, the concentration of the inhibitory agent is two times or more greater than the concentration of the unblocking agent. For example, the concentration of the inhibitory agent may be five times or more greater than the concentration of the unblocking agent or may be ten times or more greater than the concentration of the unblocking agent. In some embodiments, the concentration of the inhibitory agent is between about five times and about 10 times greater than the concentration of the unblocking agent.

FIG. 7 schematically shows conversion of an inactive form target nucleic acid 200′ that is complexed 210 with an inhibitory nucleic acid agent 220 to an active form target nucleic acid 200 that may seed on a surface of a site of an array (e.g., via hybridizing with a capture agent). The conversion is mediated by an unblocking agent that competes with the target nucleic acid 200 for binding to the inhibitory agent 220. When the unblocking agent 300 and the inhibitory agent 220 are hybridized, they form a complex 310. The conditions are tailored to provide a relatively high concentration of the complex 210 of the target nucleic acid 200′ in inactive form and the inhibitory agent 220 and a relatively low concentration of the active form target nucleic acid 200 to reduce likelihood that more than one target nucleic acid 200 seeds on the site 110 of the array such that, when amplified, a resulting cluster is monoclonal or has a high percent dominancy.

B. Encapsulating

In some embodiments, the seeding composition comprises a pool of inactive target nucleic acids encapsulated in a vehicle. The target nucleic acids are inactive because they are encapsulated and unavailable to bind a capture agent at a site of an array. The target nucleic acids may be released from the vehicle at a suitable rate to provide an appropriate concentration of active form (released) target nucleic acids for seeding. The target nucleic acids may be encapsulated in the vehicle in any suitable manner. The manner in which the target nucleic acid is encapsulated and released may depend on the vehicle employed. Release of the target nucleic acid from the vehicle may occur intrinsically in the seeding composition or may be mediated by an unblocking agent. The unblocking agent employed may depend on the vehicle employed to encapsulate the target nucleic acids.

Any suitable vehicle may be used to encapsulate the target nucleic acids. For example, the target nucleic acids may be encapsulated in a micelle, a liposome, a phage, or the like.

In some embodiments, the seeding composition comprises target nucleic acids encapsulated in a liposome. The target nucleic acids may be encapsulated in any suitable liposome in any suitable manner. For example, target nucleic acids encapsulated in liposomes by passive entrapment of active encapsulation. Passive entrapment may occur during liposome formation employing techniques such as reverse phase evaporation, dehydration/rehydration, detergent dialysis, fusion via divalent cation chelation, mixing of liposomes dissolved in an organic solvent such as ethanol and target nucleic acid dissolved in an aqueous solvent, and the like. Active encapsulation may occur by loading target nucleic acids into preformed liposomes. One example of active encapsulation is described in U.S. Pat. No. 5,227,170.

Other liposome encapsulation processes that may be used are described in U.S. Pat. No. 9,278,067; U.S. Patent Application Publication No. 2009/0068256, PCT Patent Application Publication No. WO 00/03683; U.S. Pat. No. 7,790,696; U.S. Patent Application Publication No. 2006/0058249; Jeffs et al. (March 2005), A Scalable, Extrusion-Free Method for Efficient Liposomal Encapsulation of Plasmid DNA, Pharmaceutical Research 22(3): 362-372; Gjetting et al. (May 2011), A simple protocol for preparation of a liposomal vesicle with encapsulated plasmid DNA that mediate high accumulation and reporter gene activity in tumor tissue, Results Pharma Sci. 1(1): 49-56; Fillon et al. (November 2011), Encapsulation of DNA in negatively charged liposomes and inhibition of bacterial gene expression with fluid liposome-encapsulated antisense oligonucleotides, Biochimica et Biophysica Acta (BBA)—Biomembranes 1515 (1): 44-54.

Any suitable lipid may be used to form the liposome. For example, the lipids and other molecules disclosed in the foregoing publications may be employed. In some embodiments, the liposomes comprise one or more of phosphatidyl serine, phosphatidylethanolamine, phosphatidyl choline, phosphatidylglycerol, distearoylphosphatidylcholine, distearoyl phosphatidylglycerol, and cholesterol.

The seeding solution may comprise an unblocking agent that causes the release of target nucleic acids from the liposomes. Any suitable unblocking agent may be used. For example, the unblocking agent may comprise a molecule that disrupts the liposome to release the target nucleic acid. Examples, of suitable molecules that disrupt liposomes include biological porin molecules, cytoskeletal submembranous proteins, and talin. The concentration of unblocking agent in the seeding composition may be tuned to achieve the desired concentration of active form (released) target nucleic acid in the seeding solution.

The unblocking molecule may be added to the seeding composition temporally close to the time that the seeding composition is contacted with the array or while the seeding composition is in contact with the array.

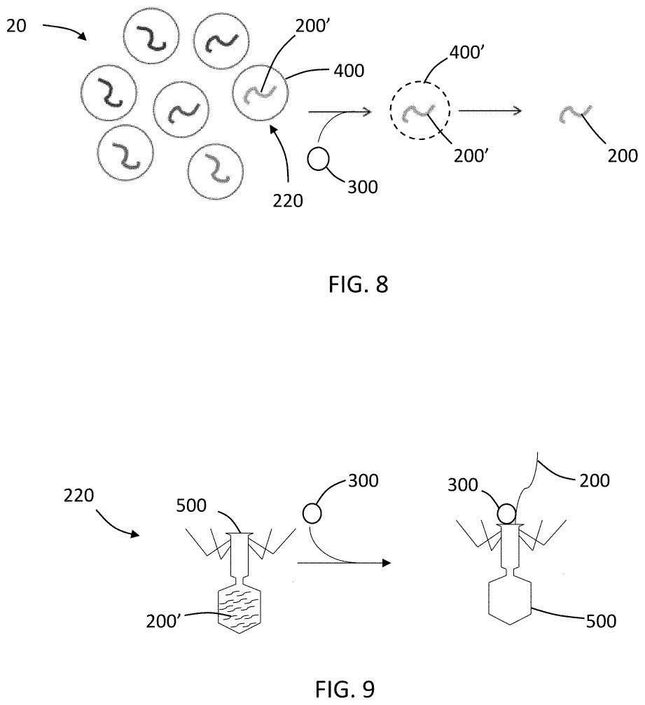

FIG. 8 schematically shows conversion of an inactive form target nucleic acid 200′ that is encapsulated in a liposome 400, which serves as an inhibitory agent 220, to an active form target nucleic acid 200. An unblocking agent 300, such as a biological porin, cytoskeletal submembranous protein, or talin, disrupts the liposome 400′ causing the release of the target nucleic acid (active form) 200. The concentration of the unblocking agent 300 may be tuned so that the rate at which the liposome 400 is disrupted is sufficiently slow to maintain a suitably low concentration of the active form target nucleic acid 200 to keep low the likelihood that more than one target nucleic acid 200 seeds on the site 110 of the array such that, when amplified, a resulting cluster is monoclonal or has a high percent dominancy.

In some embodiments, the seeding composition comprises target nucleic acids encapsulated in a viral vector such as a phage. The target nucleotides may be encapsulated in any suitable viral vector in any suitable mater. For example, target nucleic acid may be packaged into a phage by translocating the target nucleic acid into a preformed protein shell such as a prohead. The packaging may be mediated by a packaging enzyme or a terminase.

Any suitable virus or phage may be used to encapsulate the target nucleic acid. For example, adenovirus, a T4 phage, a filamentous phage, a phage lambda, or the like may be used to encapsulate the target nucleic acid.

The seeding solution may comprise an unblocking agent that causes the release of target nucleic acids from the virus or phage. Any suitable unblocking agent may be used. For example, if phage lambda is used to encapsulate the target nucleic acid, lamB may be used to cause release of the target nucleic acid from the phage. The concentration of unblocking agent in the seeding composition may be tuned to achieve the desired concentration of active form (released) target nucleic acid in the seeding solution.

The unblocking molecule may be added to the seeding composition temporally close to the time that the seeding composition is contacted with the array or while the seeding composition is in contact with the array.

One example of the use of phage to release DNA is described in Grayson et al. (September 2007), “Real-time observations of single bacteriophage λ DNA ejections in vitro”, Proc. Ntl. Acad. Sci. USA, 104(37): 14652-7, the teachings of which may be employed or modified as needed for use in seeding described herein.

FIG. 9 schematically shows conversion of an inactive form target nucleic acid 200′ that is encapsulated in a phage 500, which serves as an inhibitory agent 220, to an active form target nucleic acid 200. An unblocking agent 300, such as a lamB, interacts with the phage 500 to cause the release of the target nucleic acid (active form) 200. The concentration of the unblocking agent 300 may be tuned so that the rate at which the phage 500 releases the target nucleic acid 200 is sufficiently slow to maintain a suitably low concentration of the active form target nucleic acid 200 to keep low the likelihood that more than one target nucleic acid 200 seeds on the site 110 of the array such that, when amplified, a resulting cluster is monoclonal or has a high percent dominancy.

Inhibiting Agent as Extension of Target Nucleic Acid

When the inhibiting agent is an extension of the target nucleic acid, the inhibiting agent may hybridize to a portion of the target nucleic acid configured to bind a capture agent and thus inhibit binding of the target nucleotide to the capture agent. For example, the inhibiting agent may extend from an end of the target nucleic acid to form a hairpin loop or stem loop having a portion that hybridizes to the portion of the target nucleic acid configured to bind the capture agent. Preferably, the inhibiting agent extends from the 3′ end of the target nucleotide. The inhibiting agent may have a length of about 10 to about 100 nucleotides, about 12 to about 60 nucleotides, or about 15 to about 50 nucleotides. At least a portion of the inhibiting agent may hybridize with the portion of the target nucleic acid configured to bind the capture agent. In some embodiments, the inhibiting agent comprises a nucleotide sequence that is fully complementary to the entire portion of the target nucleic acid configured to bind the capture agent. In some embodiments, the inhibiting agent comprises a nucleotide sequence that is not fully complementary to the entire portion of the target nucleic acid configured to bind the capture agent. The inhibiting agent may comprise a nucleotide sequence that is complementary to a nucleotide sequence that is 5′ or 3′ to the portion of the target nucleic acid configured to bind the capture agent.

The percent complementarity, length of complementarity, or region of complementarity of the inhibitory agent to the target nucleic acid may be adjusted to tailor the relative affinity of the capture agent and the inhibitory agent for the target nucleic acid, which may tailor the relative concentration of inactive form target nucleic acids and active form target nucleic acids available for seeding via binding to the capture agent. In addition, the complementary portions may be modified to include non-natural nucleotides, such as locked nucleotides, that may enhance or reduce stability of base pair binding between the inhibitory agent and the target nucleic acid to alter the relative affinity of the capture agent and the inhibitory agent for the target nucleic acid.

If the inhibitory agent is fully complementary to the entire portion of the target nucleic acid configured to bind the capture agent, hybridization of the portion of the target nucleic acid configured to bind the capture agent to the inhibitory agent (intramolecular binding) may be thermodynamically favored relative to hybridization to the capture agent (intermolecular binding).

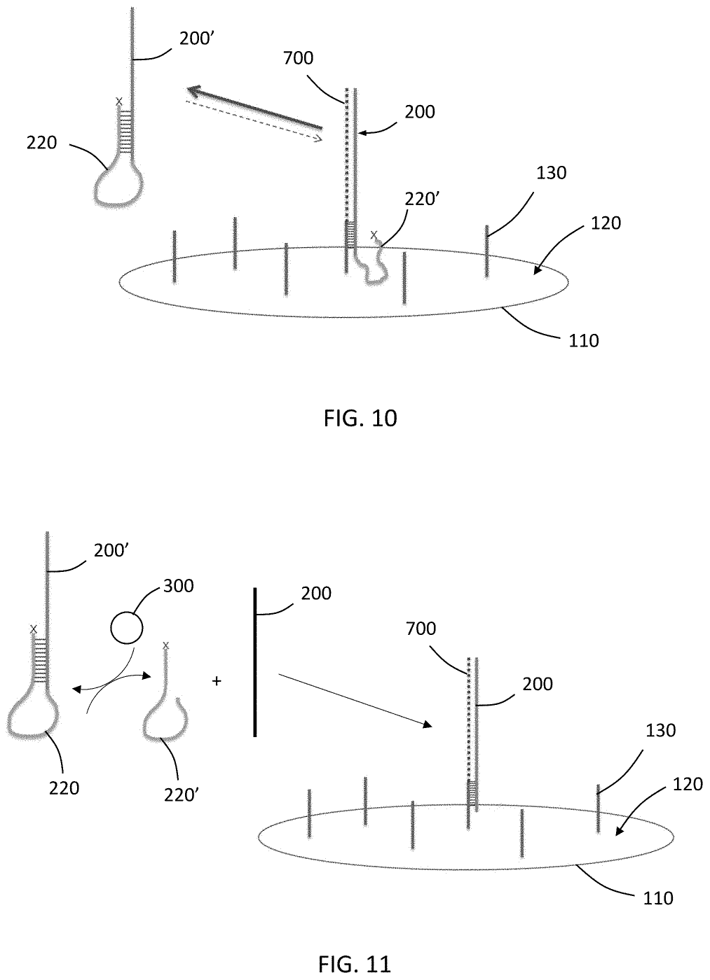

FIG. 10 schematically shows the conversion of an inactive form of a target nucleic acid 200′ to an active form target nucleic acid 200 in a seeding solution. The target nucleic acid includes a portion 220 that extends 3′ to the portion that is configured to bind a capture agent 130 on a surface 120 of a substrate at a site 110 of an array. In the inactive form 200′, the 3′ extension portion 220 hybridizes to at least a portion of the portion of the target nucleotide that is configured to bind a capture agent 130, and thus blocks binding of the target nucleic acid to the capture agent 130. In the active form 220 the 3′ extension is not hybridized to the portion of the portion of the target nucleotide that is configured to bind a capture agent 130. The active form target nucleic acid 200 bound to the capture agent 130 may be amplified to produce a copy 700 of a portion of the target nucleic acid 200. Conversion from the inactive form to the active form may occur intrinsically in the seeding composition at a rate that provides an appropriate concentration of active form target nucleic acid 200.

In some embodiments, the inhibitory agent is an extension of the target nucleic acid and the seeding composition comprises an unblocking agent that cleaves the extension of the target nucleic acid to facilitate conversion of the target nucleic acid from inactive form to active form. The extension may be cleaved by any suitable agent. For example, the extension may be cleaved by a restriction enzyme. The concentration of the restriction enzyme or other suitable cleaving agent may be controlled to tune the rate at which conversion from inactive form to active form target nucleic acid occurs.

FIG. 11 schematically shows the conversion of an inactive form of a target nucleic acid 200′ to an active form target nucleic acid 200 in a seeding solution. The target nucleic acid includes a portion 220 that extends 3′ to the portion that is configured to bind a capture agent 130 on a surface 120 of a substrate at a site 110 of an array. In the inactive form 200′, the 3′ extension portion 220 hybridizes to at least a portion of the portion of the target nucleotide that is configured to bind a capture agent 130, and thus blocks binding of the target nucleic acid to the capture agent 130. In the active form 220 the 3′ extension is not hybridized to the portion of the portion of the target nucleotide that is configured to bind a capture agent 130. The active form target nucleic acid 200 bound to the capture agent 130 may be amplified to produce a copy 700 of a portion of the target nucleic acid 200. Conversion from the inactive form to the active form is facilitated by cleaving the extension via a restriction enzyme (unblocking agent) 300.

While not shown, it will be understood that an unblocking nucleic acid agents (e.g., as depicted and described regarding FIG. 7) may be employed in methods depicted and described regarding FIG. 10 and FIG. 11.

It will be understood that the mechanisms proposed above are not exhaustive and that other suitable mechanisms for converting a pool of inactive form target nucleotides to active form target nucleotides at a rate that maintains relatively low concentrations of active nucleotide for seeding may be employed.

Library of Target Nucleic Acids

A seeding composition may comprise any suitable target nucleic acids. Preferably, the seeding composition comprises a library of different target nucleic acids derived from a sample.

Once the target nucleic acids are obtained from the sample, the target nucleic acids may be prepared for use in the methods described herein using a variety of standard techniques available and known. Exemplary methods of nucleic acid preparation include, but are not limited to, those described in Bentley et al., Nature 456:49-51 (2008); U.S. Pat. No. 7,115,400; and U.S. Patent Application Publication Nos. 2007/0128624; 2009/0226975; 2005/0100900; 2005/0059048; 2007/0110638; and 2007/0128624.

The target nucleic acids in the seeding composition may be single-stranded or double-stranded. Preferably, the target nucleic acids are double stranded. A universal adapter may be ligated to both ends of the double-stranded target nucleic acids derived from the sample. The universal adapter may include a region of double stranded nucleic acid and a region of single-stranded non-complementary nucleic acid strands. The region of single-stranded non-complementary nucleic acid strands may include at the 3′ ends a first universal capture binding sequence. The first universal capture binding sequence may bind to at least a portion of a first capture agent having a sequence of nucleotides complementary to the first universal capture binding sequence. The first capture agent may be at a site of an array.

The region of single-stranded non-complementary nucleic acid strands may optionally include at the 5′ end a second universal capture binding sequence. The second universal capture binding sequence may bind to at least a portion of a second capture agent having a sequence of nucleotides complementary to the second universal capture binding sequence. The second capture agent may be at the site of the array to allow for bridge amplification of the target nucleic acid.

The universal adapter may be ligated to double stranded target nucleic acids using any suitable process, such as ligation methods known in the art. Such methods use ligase enzymes such as DNA ligase to effect or catalyze joining of the ends of the two nucleic acid strands of, in this case, the universal adapter and the double-stranded target nucleic acids, such that covalent linkages are formed. The universal adapter may contain a 5′-phosphate moiety to facilitate ligation to the 3′-OH present on the target fragment. The double-stranded target nucleic acid contains a 5′-phosphate moiety, either residual from the shearing process, or added using an enzymatic treatment step, and has been end repaired, and optionally extended by an overhanging base or bases, to give a 3′-OH suitable for ligation. In this context, joining means covalent linkage of nucleic acid strands which were not previously covalently linked. In some aspects of the disclosure, such joining takes place by formation of a phosphodiester linkage between the two nucleic acid strands, but other means of covalent linkage (e.g. non-phosphodiester backbone linkages) may be used.

The universal adapters used in the ligation may include one or more universal capture binding sequence and other universal sequences, such as a universal primer binding site and an index sequence. The target nucleic acids may be used for seeding and amplification and subsequently for sequencing as described herein.

The target nucleic acids may also be modified to include any suitable nucleotide sequence using standard, known methods. Such sequences may include, for example, restriction enzyme sites, or indexing tags in order to permit identification of amplification products of a given nucleic acid sequence.

Amplification/Cluster Formation

Once the target nucleic acid is seeded on a substrate at a site of an array, the target nucleic acid may be amplified to produce a cluster at the site. Reagents suitable for amplifying the target nucleic acid are preferably be included in the seeding composition.

A method of the present disclosure may include amplifying a target nucleic acid to produce an amplification site that includes a monoclonal population of amplicons from an individual target nucleic acid that has seeded the site or that includes a high percent dominancy of the individual target nucleic acid at the site. The rate of target nucleic acid seeding is kept low by keeping the concentration of active form target nucleic acid low, e.g. as described above.

Exclusion amplification occurs due to the relatively slow rate of target nucleic acid seeding vs. the relatively rapid rate at which amplification occurs to fill the site with copies of the seeded nucleic acid. Once a first target nucleic acid begins amplification, the site will rapidly fill to capacity or near capacity with its copies, thereby inhibiting seeding of a second target nucleic acid at the site.

Even if an amplification site is not filled to capacity prior to seeding and amplification of a second target nucleic acid at the site, the amount of the first nucleic acid may dominate the amount of the second nucleic acid such that, when sequencing occurs, the signal from the first nucleic acid may be differentiated from, or dominate, the signal from the second nucleic acid, and the sequence from the first nucleic acid may be determined. By way of example, when 14 cycles of exponential bridge amplification are performed on a circular site smaller than 500 nm in diameter, contamination from seeding and amplification from a second target nucleic acid will produce an insufficient number of contaminating amplicons to adversely impact sequencing-by-synthesis analysis on an Illumina sequencing platform.

Preferably, the amplifications sites in an array are monoclonal or comprise a dominant target nucleic acid amplicon having a sufficiently low level of contaminating amplicons from a second target nucleic acid such that the level of contamination does not have an unacceptable impact on a subsequent use of the array. For example, when the array is to be used in a detection application, an acceptable level of contamination would be a level that does not impact signal to noise or resolution of the detection technique in an unacceptable way. Illustrative levels of contamination that may be acceptable at an individual amplification site for particular applications include, but are not limited to, at most 0.1%, 0.5%, 1%, 5%, 10% or 25% contaminating amplicons. An array may include one or more amplification sites having these illustrative levels of contaminating amplicons. For example, up to 5%, 10%, 25%, 50%, 75%, or even 100% of the amplification sites in an array may have some contaminating amplicons.

The methods described herein may be carried out under conditions wherein the target nucleic acids are transported (e.g. via diffusion) to the amplification sites as amplification is occurring. Thus, exclusion amplification may exploit a relatively slow transport rate due to relatively low concentrations of active form target nucleic acid that may seed. Thus, an amplification reaction set forth herein may be carried out such that target nucleic acids are transported from solution to amplification sites simultaneously with (i) the producing of a first amplicon, and (ii) the producing of the subsequent amplicons at other sites of the array. The average rate at which the amplicons are generated at the amplification sites (i.e., the rate of amplification) may exceed the average rate at which the target nucleic acids are transported from the solution to the amplification sites (i.e., the rate of seeding). In some cases, a sufficient number of amplicons may be generated from a single target nucleic acid at an individual amplification site to fill the capacity of the respective amplification site. The rate at which amplicons are generated to fill the capacity of respective amplification sites may, for example, exceed the rate at which the individual target nucleic acids are transported from the solution to the amplification sites.

An amplification composition that is used in a method set forth herein is preferably capable of rapidly making copies of target nucleic acids at amplification sites. Typically, an amplification composition used in a method of the present disclosure will include a polymerase and nucleotide triphosphates (NTPs). Any of a variety of polymerases known in the art may be used, but in some embodiments, it may be preferable to use a polymerase that is exonuclease negative. The NTPs may be deoxyribonucleotide triphosphates (dNTPs) for embodiments where DNA copies are made. Typically, the four native species, dATP, dTTP, dGTP and dCTP, will be present in a DNA amplification reagent; however, analogs may be used if desired. The NTPs may be ribonucleotide triphosphates (rNTPs) for embodiments where RNA copies are made. Typically, the four native species, rATP, rUTP, rGTP and rCTP, will be present in an RNA amplification reagent; however, analogs may be used if desired.

An amplification composition, which may be the seeding composition, may include further components that facilitate amplicon formation and, in some cases, increase the rate of amplicon formation. An example is a recombinase loading protein. Recombinase may facilitate amplicon formation by allowing repeated invasion/extension. More specifically, recombinase may facilitate invasion of a target nucleic acid by the polymerase and extension of a primer by the polymerase using the target nucleic acid as a template for amplicon formation. This process may be repeated as a chain reaction where amplicons produced from each round of invasion/extension serve as templates in a subsequent round. The process may occur more rapidly than standard PCR since a denaturation cycle (e.g. via heating or chemical denaturation) is not required. As such, recombinase-facilitated amplification may be carried out isothermally. It is generally desirable to include ATP, or other nucleotides (or in some cases non hydrolysable analogs thereof) in a recombinase-facilitated amplification composition to facilitate amplification. A mixture of recombinase, single stranded binding (SSB) protein, and accessory protein is particularly useful. Illustrative formulations for recombinase-facilitated amplification include those sold commercially as TwistAmp kits by TwistDx (Cambridge, UK). Useful components of recombinase-facilitated amplification composition and reaction conditions are set forth in U.S. Pat. Nos. 5,223,414 and 7,399,590.

Another example of a component that may be included in an amplification composition to facilitate amplicon formation and in some cases to increase the rate of amplicon formation is a helicase. Helicase may facilitate amplicon formation by allowing a chain reaction of amplicon formation. The process may occur more rapidly than standard PCR since a denaturation cycle (e.g. via heating or chemical denaturation) is not required. As such, helicase-facilitated amplification may be carried out isothermally. A mixture of helicase and single stranded binding (SSB) protein is particularly useful as SSB may further facilitate amplification. Illustrative formulations for helicase-facilitated amplification include those sold commercially as IsoAmp kits from Biohelix (Beverly, Mass.). Further, examples of useful formulations that include a helicase protein are described in U.S. Pat. Nos. 7,399,590 and 7,829,284.

Yet another example of a component that may be included in an amplification composition to facilitate amplicon formation and in some cases increase the rate of amplicon formation is an origin binding protein.

The presence of molecular crowding reagents in the amplification composition may be used to aid exclusion amplification. Examples of useful molecular crowding reagents include, but are not limited to, polyethylene glycol (PEG), Ficoll®, dextran, or polyvinyl alcohol. Illustrative molecular crowding reagents and formulations are set forth in U.S. Pat. No. 7,399,590.

The rate at which an amplification reaction occurs may be increased by increasing the concentration or amount of one or more of the active components of an amplification reaction. For example, the amount or concentration of polymerase, nucleotide triphosphates, primers, recombinase, helicase or SSB may be increased to increase the amplification rate. In some cases, the one or more active components of an amplification reaction that are increased in amount or concentration (or otherwise manipulated in a method set forth herein) are non-nucleic acid components of the amplification reaction.

Amplification rate may also be increased by adjusting the temperature. For example, the rate of amplification at one or more amplification sites may be increased by increasing the temperature at the site(s) up to a maximum temperature where reaction rate declines due to denaturation or other adverse events. Optimal or desired temperatures may be determined from known properties of the amplification components in use or empirically for a given amplification reaction mixture. Such adjustments may be made based on a priori predictions of primer melting temperature (Tm) or empirically.

The rate at which an amplification reaction occurs may be increased by increasing the activity of one or more amplification reagent. For example, a cofactor that increases the extension rate of a polymerase may be added to a reaction where the polymerase is in use. In some embodiments, metal cofactors such as magnesium, zinc or manganese may be added to a polymerase reaction or betaine may be added.

In some embodiments, a population of target nucleic acids that is double-stranded is used. It has been observed that amplicon formation at an array of sites under exclusion amplification conditions is efficient for double-stranded target nucleic acids. For example, a plurality of amplification sites having populations of amplicons may be more efficiently produced from double-stranded target nucleic acids (compared to single-stranded target nucleic acids at the same concentration) in the presence of recombinase and single-stranded binding protein. Nevertheless, it will be understood that single-stranded target nucleic acids may be used in some embodiments of the methods set forth herein.

A method set forth herein may use any of a variety of amplification techniques. Illustrative techniques that may be used include, but are not limited to, polymerase chain reaction (PCR), rolling circle amplification (RCA), multiple displacement amplification (MDA), or random prime amplification (RPA). In some embodiments the amplification may be carried out in solution, for example, when the amplification sites are capable of containing amplicons in a volume having a desired capacity. Preferably, an amplification technique used under conditions of exclusion amplification in a method of the present disclosure will be carried out on solid phase. For example, one or more primers used for amplification may be attached to a solid phase at the amplification site. As discussed above capture agents for seeding may comprise the one or more primers. In PCR embodiments, one or both of the primers used for amplification may be attached to a solid phase. Formats that utilize two species of primer attached to the surface are often referred to as bridge amplification because double stranded amplicons form a bridge-like structure between the two surface-attached primers that flank the template sequence that has been copied. Illustrative reagents and conditions that may be used for bridge amplification are described, for example, in U.S. Pat. No. 5,641,658; U.S. Pat. Pub. No. 2002/0055100; U.S. Pat. No. 7,115,400; U.S. Pat. Pub. No. 2004/0096853; U.S. Pat. Pub. No. 2004/0002090; U.S. Pat. Pub. No. 2007/0128624; and U.S. Pat. Pub. No. 2008/0009420. Solid-phase PCR amplification may also be carried out with one of the amplification primers attached to a solid support and the second primer in solution. An illustrative format that uses a combination of a surface attached primer and soluble primer is emulsion PCR as described, for example, in Dressman et al., Proc. Natl. Acad. Sci. USA 100:8817-8822 (2003), WO 05/010145, or U.S. Pat. Pub. Nos. 2005/0130173 or 2005/0064460. Emulsion PCR is illustrative of the format and it will be understood that for purposes of the methods set forth herein the use of an emulsion is optional and indeed for several embodiments an emulsion is not used. The described PCR techniques may be modified for non-cyclic amplification (e.g. isothermal amplification) using components exemplified elsewhere herein for facilitating or increasing the rate of amplification. Accordingly, the described PCR techniques may be used under exclusion amplification conditions.

RCA techniques may be modified for use in a method of the present disclosure. Illustrative components that may be used in an RCA reaction and principles by which RCA produces amplicons are described, for example, in Lizardi et al., Nat. Genet. 19:225-232 (1998) and US 2007/0099208 A1. Primers used for RCA may be in solution or attached to a solid support surface at an amplification site. The RCA techniques exemplified in the above references may be modified in accordance with teaching herein, for example, to increase the rate of amplification to suit particular applications. Thus, RCA techniques may be used under exclusion amplification conditions.

MDA techniques may be modified for use in a method of the present disclosure. Some basic principles and useful conditions for MDA are described, for example, in Dean et al., Proc Natl. Acad. Sci. USA 99:5261-66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); U.S. Pat. Nos. 5,455,166; 5,130,238; and 6,214,587. Primers used for MDA may be in solution or attached to a solid support surface at an amplification site. The MDA techniques exemplified in the above references may be modified in accordance with teaching herein, for example, to increase the rate of amplification to suit particular applications. Accordingly, MDA techniques may be used under exclusion amplification conditions.

A combination of the described amplification techniques may be used to make an array under exclusion amplification conditions. For example, RCA and MDA may be used in a combination wherein RCA is used to generate a concatemeric amplicon in solution (e.g. using solution-phase primers). The amplicon may then be used as a template for MDA using primers that are attached to a solid support surface at an amplification site. In this example, amplicons produced after the combined RCA and MDA steps will be attached to the surface of the amplification site.

As exemplified with respect to several of the embodiments above, a method of the present disclosure need not use a cyclical amplification technique. For example, amplification of target nucleic acids may be carried out at amplification sites absent a denaturation cycle. Illustrative denaturation cycles include introduction of chemical denaturants to an amplification reaction and/or increasing the temperature of an amplification reaction. Thus, amplifying of the target nucleic acids need not include a step of replacing the amplification solution with a chemical reagent that denatures the target nucleic acids and the amplicons. Similarly, amplifying of the target nucleic acids need not include heating the solution to a temperature that denatures the target nucleic acids and the amplicons. Accordingly, amplifying of target nucleic acids at amplification sites may be carried out isothermally for the duration of a method set forth herein. Indeed, an amplification method set forth herein may occur without one or more cyclic manipulations that are carried out for some amplification techniques under standard conditions. Furthermore, in some standard solid phase amplification techniques a wash is carried out after target nucleic acids are loaded onto a substrate and before amplification is initiated. However, in embodiments of the present methods, a wash step need not be carried out between transport of target nucleic acids to reaction sites and amplification of the target nucleic acids at the amplification sites. Instead, transport (e.g. via diffusion) and amplification are allowed to occur simultaneously to provide for exclusion amplification.

In some embodiments, it may be desirable to repeat an amplification cycle that occurs under exclusion amplification conditions. Thus, although copies of a target nucleic acid may be made at an individual amplification site without cyclic manipulations, an array of amplification sites may be treated cyclically to increase the number of sites that contain amplicons after each cycle. In particular embodiments, the amplification conditions may be modified from one cycle to the next. For example, one or more of the conditions set forth above for altering the rate of transport or altering the rate of amplification may be adjusted between cycles. As such, the rate of transport may be increased from cycle to cycle, the rate of transport may be decreased from cycle to cycle, the rate of amplification may be increased from cycle to cycle, or the rate of amplification may be decreased from cycle to cycle.

Use in Sequencing/Methods of Sequencing

An array of the present disclosure, for example, having been produced by a seeding and amplification method set forth herein to produce amplified target nucleic acids at amplification sites, may be used for any of a variety of applications. A particularly useful application is nucleic acid sequencing. One example is sequencing-by-synthesis (SBS). In SBS, extension of a nucleic acid primer along a nucleic acid template (e.g., a target nucleic acid or amplicon thereof) is monitored to determine the sequence of nucleotides in the template. The underlying chemical process may be polymerization (e.g., as catalyzed by a polymerase enzyme). In a particular polymerase-based SBS embodiment, fluorescently labeled nucleotides are added to a primer (thereby extending the primer) in a template dependent fashion such that detection of the order and type of nucleotides added to the primer may be used to determine the sequence of the template. A plurality of different templates at different sites of an array set forth herein may be subjected to an SBS technique under conditions where events occurring for different templates may be distinguished due to their location in the array.

Flow cells provide a convenient format for housing an array that is produced by the methods of the present disclosure and that is subjected to an SBS or other detection technique that involves repeated delivery of reagents in cycles. For example, to initiate a first SBS cycle, one or more labeled nucleotides, DNA polymerase, etc., may be flowed into/through a flow cell that houses an array of nucleic acid templates. Those sites of an array where primer extension causes a labeled nucleotide to be incorporated may be detected. Optionally, the nucleotides may further include a reversible termination property that terminates further primer extension once a nucleotide has been added to a primer. For example, a nucleotide analog having a reversible terminator moiety may be added to a primer such that subsequent extension cannot occur until a deblocking agent is delivered to remove the moiety. Thus, for embodiments that use reversible termination, a deblocking reagent may be delivered to the flow cell (before or after detection occurs). Washes may be carried out between the various delivery steps. The cycle may then be repeated n times to extend the primer by n nucleotides, thereby detecting a sequence of length n. Illustrative SBS procedures, fluidic systems and detection platforms that may be readily adapted for use with an array produced by the methods of the present disclosure are described, for example, in Bentley et al., Nature 456:53-59 (2008), WO 04/018497; U.S. Pat. No. 7,057,026; WO 91/06678; WO 07/123,744; U.S. Pat. Nos. 7,329,492; 7,211,414; 7,315,019; 7,405,281, and 8,343,746.

Other sequencing procedures that use cyclic reactions may be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320). In pyrosequencing, released PPi may be detected by being immediately converted to adenosine triphosphate (ATP) by ATP sulfurylase, and the level of ATP generated may be detected via luciferase-produced photons. Thus, the sequencing reaction may be monitored via a luminescence detection system. Excitation radiation sources used for fluorescence-based detection systems are not necessary for pyrosequencing procedures. Useful fluidic systems, detectors and procedures that may be used for application of pyrosequencing to arrays of the present disclosure are described, for example, in WIPO Published Pat. App. 2012/058096, US 2005/0191698 A1, U.S. Pat. Nos. 7,595,883, and 7,244,559.

Sequencing-by-ligation reactions are also useful including, for example, those described in Shendure et al. Science 309:1728-1732 (2005); U.S. Pat. Nos. 5,599,675; and 5,750,341. Some embodiments may include sequencing-by-hybridization procedures as described, for example, in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977. In both sequencing-by-ligation and sequencing-by-hybridization procedures, target nucleic acids (e.g., a target nucleic acid or amplicons thereof) that are present at sites of an array are subjected to repeated cycles of oligonucleotide delivery and detection. Fluidic systems for SBS methods as set forth herein or in references cited herein may be readily adapted for delivery of reagents for sequencing-by-ligation or sequencing-by-hybridization procedures. Typically, the oligonucleotides are fluorescently labeled and may be detected using fluorescence detectors similar to those described with regard to SBS procedures herein or in references cited herein.

Some embodiments may use methods involving the real-time monitoring of DNA polymerase activity. For example, nucleotide incorporations may be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008).

Some SBS embodiments include detection of a proton released upon incorporation of a nucleotide into an extension product. For example, sequencing based on detection of released protons may use an electrical detector and associated techniques that are commercially available from Ion Torrent (Guilford, Conn., a Life Technologies subsidiary) or sequencing methods and systems described in US 2009/0026082 A1; US 2009/0127589 A1; US 2010/0137143 A1; or US 2010/0282617 A1. Methods set forth herein for seeding and amplifying target nucleic acids may be readily applied to substrates used for detecting protons. More specifically, methods set forth herein may be used to produce clonal populations of amplicons at the sites of the arrays that are used to detect protons.

A useful application for an array of the present disclosure, for example, having been produced by a method set forth herein, is gene expression analysis. Gene expression may be detected or quantified using RNA sequencing techniques, such as those referred to as digital RNA sequencing. RNA sequencing techniques may be carried out using sequencing methodologies known in the art such as those set forth above. Gene expression may also be detected or quantified using hybridization techniques carried out by direct hybridization to an array or using a multiplex assay, the products of which are detected on an array. An array of the present disclosure, for example, having been produced by a method set forth herein, may also be used to determine genotypes for a genomic DNA sample from one or more individual. Illustrative methods for array-based expression and genotyping analysis that may be carried out on an array of the present disclosure are described in U.S. Pat. Nos. 7,582,420; 6,890,741; 6,913,884 or 6,355,431 or US Pat. Pub. Nos. 2005/0053980 A1; 2009/0186349 A1 or US 2005/0181440 A1.

Another useful application for an array having been produced by a method set forth herein is single-cell sequencing. When combined with indexing methods single cell sequencing may be used in chromatin accessibility assays to produce profiles of active regulatory elements in thousands of single cells, and single cell whole genome libraries may be produced. Examples for single-cell sequencing that may be carried out on an array of the present disclosure are described in U.S. Published Patent Application 2018/0023119 A1, U.S. Provisional Application Ser. Nos. 62/673,023 and 62/680,259.

An advantage of the methods set forth herein is that they provide for rapid and efficient creation of arrays from any of a variety of nucleic acid libraries. Accordingly, the present disclosure provides integrated systems capable of making an array using one or more of the methods set forth herein and further capable of detecting nucleic acids on the arrays using techniques known in the art such as those exemplified above. Thus, an integrated system of the present disclosure may include fluidic components capable of delivering amplification reagents to an array of amplification sites such as pumps, valves, reservoirs, fluidic lines and the like. A particularly useful fluidic component is a flow cell. A flow cell may be configured and/or used in an integrated system to create an array of the present disclosure and to detect the array. Illustrative flow cells are described, for example, in US 2010/0111768 A1 and U.S. Pat. No. 8,951,781. As exemplified for flow cells, one or more of the fluidic components of an integrated system may be used for an amplification method and for a detection method. Taking a nucleic acid sequencing embodiment as an example, one or more of the fluidic components of an integrated system may be used for an amplification method set forth herein and for the delivery of sequencing reagents in a sequencing method such as those exemplified above. Alternatively, an integrated system may include separate fluidic systems to carry out amplification methods and to carry out detection methods. Examples of integrated sequencing systems that are capable of creating arrays of nucleic acids and also determining the sequence of the nucleic acids include, without limitation, the MiSeq™ and HiSeq™ platform (Illumina, Inc., San Diego, Calif.) and devices described in U.S. Pat. No. 8,951,781. Such devices may be modified to make arrays using exclusion amplification in accordance with the guidance set forth herein.

A system capable of carrying out a method set forth herein need not be integrated with a detection device. Rather, a stand-alone system or a system integrated with other devices is also possible. Fluidic components similar to those exemplified above in the context of an integrated system may be used in such embodiments.

A system capable of carrying out a method set forth herein, whether integrated with detection capabilities or not, may include a system controller that is capable of executing a set of instructions to perform one or more steps of a method, technique or process set forth herein. For example, the instructions may direct the performance of steps for creating an array under exclusion amplification conditions. Optionally, the instructions may further direct the performance of steps for detecting nucleic acids using methods set forth previously herein. A useful system controller may include any processor-based or microprocessor-based system, including systems using microcontrollers, reduced instruction set computers (RISC), application specific integrated circuits (ASICs), field programmable gate array (FPGAs), logic circuits, and any other circuit or processor capable of executing functions described herein. A set of instructions for a system controller may be in the form of a software program. As used herein, the terms “software” and “firmware” are interchangeable and include any computer program stored in memory for execution by a computer, including RAM memory, ROM memory, EPROM memory, EEPROM memory, and non-volatile RAM (NVRAM) memory. The software may be in various forms such as system software or application software. Further, the software may be in the form of a collection of separate programs, or a program module within a larger program or a portion of a program module. The software also may include modular programming in the form of object-oriented programming.

### EXAMPLES

The present invention is illustrated by the following non-limiting examples. It is to be understood that the particular examples, materials, amounts, and procedures are to be interpreted broadly in accordance with the scope and spirit of the invention as set forth herein.

### Example 1

To illustrate the polyclonality of clusters obtained by currently employed technologies, patterned clusters were grown using Illumina's SBS technology.

FIG. 12 is a stochastic optical reconstruction microscopy (STORM) image of 550 nm pitch, 360 nm diameter nanowell clusters. Different colors emanated from many of the clusters, indicating that the clusters are polyclonal.

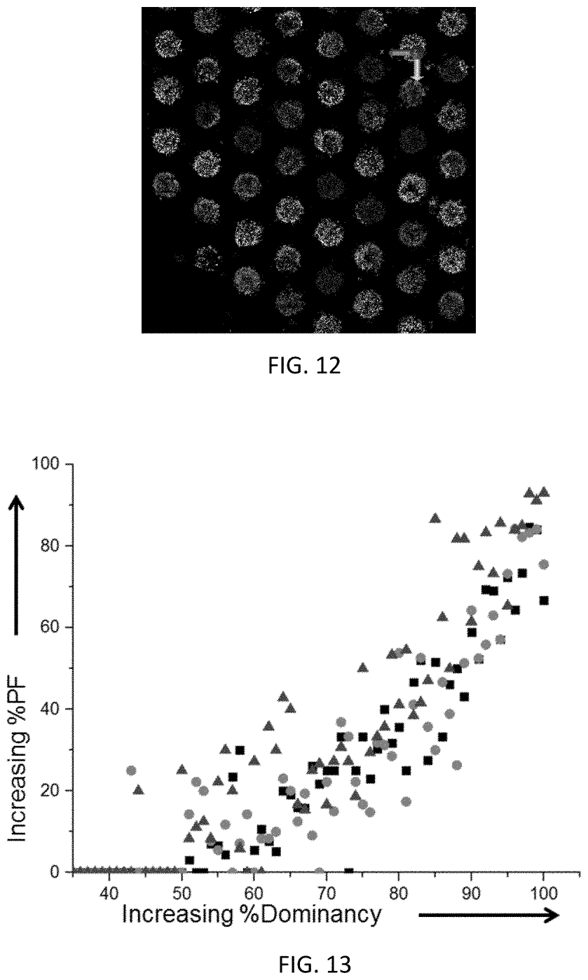

Many of the polyclonal clusters are sequenceable, because even though several reads (target nucleic acids) are present, one tends to “dominate”. Such clusters may be said to have high “dominancy”. A cluster that is able to be sequenced is said be “Passing filters” or PF. We have found a strong correlation between the % Dominancy of a cluster and the percentage of those clusters that PF. As shown in FIG. 13, as clusters increase in % Dominancy, they also increase in % PF. Each data point in FIG. 13 represents the mean % Dominancy determined by STORM imaging and mean % PF, determined by sequencing, of a population of several 10s to 100s of nanowells.

During a sequencing run, it is desirable to have the highest possible % of clusters PF, so that the throughput of the run is maximized and is the most efficient. In current sequencers, using patterned flow-cells, the % PF is typically in the 60-80% range, and therefore 20% to 40% of the clusters are not useable for sequencing. If the clusters were grown so that more had the highest dominancies (ideally of 100% dominancy), then an increased % PF would be realized, which would increase throughput without additional flow-cells, reagents, or run-time.

### Example 2

One way to improve monoclonality or increase % Dominancy it to reduce the concentration of target nucleic acid in a sample for seeding and amplification and perform repeated seed/amplification steps. Polyclonality occurs in a patterned nanowell flow cell when multiple target nucleic acids land in a nanowell at a time and are amplified. The probability of this occurring is very much dependent on the target nucleic acid concentration. At low concentration, a situation may be achieved in which the nanowells are highly monoclonal or have a high % Dominancy. However, only a fraction of the nanowells may grow clusters during a single round of seeding an amplification when low target nucleic acid concentrations are employed, which results in low overall throughput of the flowcell.

To improve the number of nanowells in which clusters grow, repeat or continuous seed and amplifications steps may be performed. For those wells in which a cluster has already grown in a previous round of seed and amplification, further seeding by additional target nucleic acids in a subsequent round is not problematic because the first seeded nucleic acid will be dominant or there will be very few binding sites available for the additional target nucleic acid to bind, which will reduce the likelihood of binding in a cluster having a previously seeded and amplified target nucleic acid.

In this example, five sequential seed and amplification steps were performed. The first step employed 20 pM target DNA to produce red clusters, the second step employed 20 pM target DNA to produce Green clusters, the third step employed 20 pM target DNA to produce White clusters, the fourth step employed 20 pM target DNA to produce Magenta clusters, and the fifth step employed 200 pM target DNA to produce yellow clusters. During each step, amplification mix was incubated for 15 minutes and then flushed with buffer.

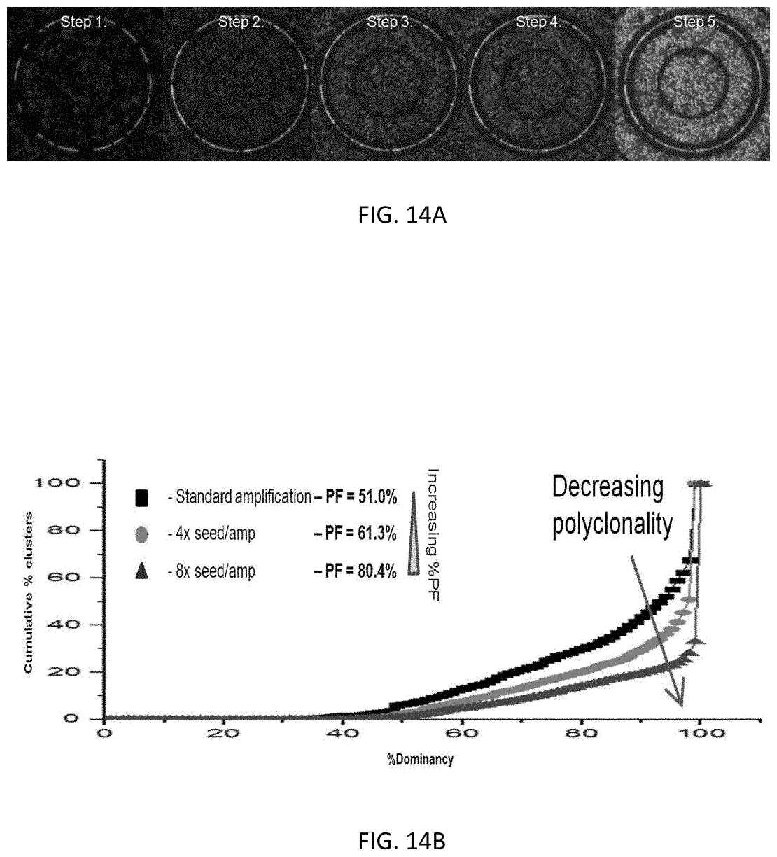

The results are shown in FIG. 14A, in which STORM images following each of the five sequential steps are shown from left to right. As shown, highly dominant or monoclonal clusters were produced with a high percentage of nanowells containing clusters.

Analysis of the STORM images was done to determine dominancy of the clusters. Results are shown in FIG. 14B, which demonstrates that the repeated low concentration seed and amplification strategy produces a high % Dominancy. Sequencing was performed We also confirmed, by sequencing, that this leads to higher % PF overall.

The % PF following standard (single step) seeding (300 pm) and amplification was 51.0%. The % PF following four rounds of sequential low concentration (20 pM) seeding and amplification was 61.3%. The % PF following eight rounds of sequential low concentration (20 pM) seeding and amplification was 80.4%.

These results clearly show that the repeat low concentration seed and amp strategy does achieve the desired effect. However, this process has several drawbacks. For example, it requires repeated steps, takes more time, and uses substantially more amplification reagents.

### Example 3

In this prophetic example, a process referred to herein as Template Controlled-Release And Seeding—TECRAS is described. TECRAS may address shortcomings associated with the repeated low concentration seed and amplification discussed above. For example, TECRAS may involving only a single step of seeding and amplification to achieve similar goals of higher % Dominancy and higher % PF.

In this method the DNA target library is present in the amplification mix at high concentration (e.g. 300 pM). However, it is present in an initially inactive form that cannot seed. A slow process then occurs that converts these DNA molecules from the inactive to an active form which may seed and begin amplification. The rate of this process is tuned such that at any given moment, the concentration of Active DNA is low (e.g. 20 pM). This has the same effect as repeated seed and amplification but has the benefit that this is a single incubation step.

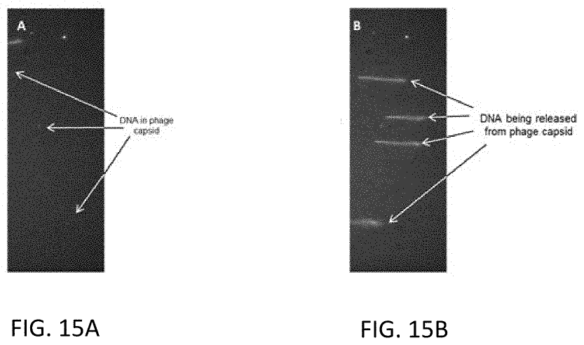

To illustrate the concept of using phages to hold a pool of inactive form target nucleic acid and release active form target nucleic acid for seeding, DNA was incorporated into lambda phage and controlled release was achieved by the addition of LamB. The results are presented in FIGS. 15A-B. DNA was held securely within the phage capsid (15A) until the addition of the LamB trigger protein caused phage capsid to release the DNA into solution (15B). FIG. 15A shows some DNA held on the surface within the phage, and FIG. 15B shows the same area some time later after LamB caused DNA molecules to be released from the phage.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising:
providing a substrate having a surface to which a capture agent is bound;
providing a composition comprising (i) a plurality of different target nucleic acids, each comprising a universal sequence, and (ii) an inhibiting agent that inhibits binding of at least a portion of the universal sequence to the capture agent;
contacting the surface of the substrate with the composition to bind one of the target nucleic acids to the capture agent; and
amplifying the target nucleic acid bound to the capture agent while the composition is in contact with the surface of the substrate.

2. The method of claim 1, wherein the capture agent comprises a nucleic acid having a nucleotide sequence, and wherein the universal sequence comprises a nucleotide sequence complementary to at least a portion of the nucleotide sequence of the nucleic acid of the capture agent.

3. The method of claim 2, where the inhibiting agent comprises a nucleic acid having a nucleotide sequence that is the same as at least the portion of the nucleotide sequence of the nucleic acid of the capture agent.

4. The method of claim 3, wherein the composition further comprises an unblocking agent that comprises a nucleic acid having a nucleotide sequence complementary to at least a portion of the nucleic acid of the inhibiting agent.

5. The method of claim 4, wherein the concentration of the unblocking agent in the composition is lower than the concentration of the inhibiting agent.

6. The method of claim 1, wherein the inhibiting agent encapsulates the plurality of different target nucleic acids.

7. The method of claim 6, wherein the inhibiting agent comprises a liposome.

8. The method of claim 6, wherein the inhibiting agent comprises a phage.

9. The method of claim 6, wherein the composition further comprises an unblocking agent configured to release the plurality of different target nucleic acids encapsulated in the inhibiting agent.

10. The method of claim 9, wherein the inhibiting agent comprises a liposome and the unblocking agent comprises a molecule configured to disrupt a membrane of the liposome to release the plurality of different target nucleic acids from the liposome.

11. The method of claim 10, wherein the unblocking agent comprises a porin, talin, or a cytoskeletal submembranous protein.

12. The method of claim 9, wherein the inhibiting agent comprises a bacteriophage lambda and wherein the unblocking agent comprises lamB.

13. The method of claim 1, wherein the composition is configured to provide a concentration of target nucleic acids available to bind to the capture agents in a range from about 5 picomolar (pM) to about 50 pM during a time in which the composition is contacted with the surface of the substrate.

14. The method of claim 1, wherein the plurality of different target nucleic acids comprises DNA.

15. The method of claim 1, wherein the capture agent is a part of an array of capture agents.

16. The method of claim 1, wherein the substrate is part of a sequencing flow cell.

17. A method comprising:
providing a substrate having a surface to which a capture agent is bound;
providing a composition comprising a plurality of different target nucleic acids, each comprising a universal sequence configured to bind the capture agent, wherein the universal sequence of at least some of the plurality of different nucleic acids are blocked from binding to the capture agent;
contacting the surface of the substrate with the composition;
unblocking the universal sequence of at least some of the plurality of different target nucleic acids to allow the unblocked nucleic acids to bind to the capture agent;
amplifying the nucleic acids that are bound to the capture agent while the composition is in contact with the surface of the substrate.

18. The method of claim 17, wherein the composition is configured to provide a concentration of target nucleic acids available to bind to the capture agents in a range from about 5 picomolar (pM) to about 50 pM during a time in which the composition is contacted with the surface of the substrate.

19. The method of claim 17, wherein the plurality of different target nucleic acids comprises DNA.

20. The method of claim 17, wherein the capture agent is a part of an array of capture agents.
